(19) United States
(12) Patent Application Publication
(10) Pub. No.: US 2013/0337034 A1
(43) Pub. Date: Dec. 19, 2013

(54) BIOFUNCTIONALIZED MAGNETIC NANOWIRES

(71) Applicants: Jürgen Kosel, Thuwal (SA); Timothy Ravasi, Thuwal (SA); Maria Fernanda Contreras Gerenas, Thuwal (SA)

(72) Inventors: Jürgen Kosel, Thuwal (SA); Timothy Ravasi, Thuwal (SA); Maria Fernanda Contreras Gerenas, Thuwal (SA)

(73) Assignee: KING ABDULLAH UNIVERSITY OF SCIENCE AND TECHNOLOGY, Thuwal (SA)

(21) Appl. No.: 13/919,552

(22) Filed: Jun. 17, 2013

Related U.S. Application Data
(60) Provisional application No. 61/660,929, filed on Jun. 18, 2012.

(51) Int. Cl. A61K 41/00

(52) U.S. Cl. CPC A61K 41/0052
USPC 424/443; 530/391.1; 424/178.1

(57) ABSTRACT

Magnetic nanowires can be used as an alternative method overcoming the limitations of current cancer treatments that lack specificity and are highly cytotoxic. Nanowires are developed so that they selectively attach to cancer cells via antibodies, potentially destroying them when a magnetic field induces their vibration. This will transmit a mechanical force to the targeted cells, which is expected to induce apoptosis on the cancer cells.

### CLAIM OF PRIORITY

This application claims the benefit of prior U.S. Provisional Application No. 61/660,929, filed on Jun. 18, 2012, which is incorporated by reference in its entirety.

### TECHNICAL FIELD

This invention relates to biological uses of nanowires.

### BACKGROUND

There are many advantages to being able to manipulate cells. For example, cancer cells can be manipulated to undergo apoptosis. Cancer in its different forms has become one of the major diseases with huge social and economic impact. In the US only, the cost associated with cancer diagnosis, treatment and follow-up was about $125 billion in 2010 and this number is expected to increase quickly. Typical cancer treatments include chemotherapy, radiotherapy, and surgery. These therapies currently encounter challenges such as non-specific delivery of antitumor agents, inadequate drug concentrations reaching the tumor, and a limited capability of monitoring therapeutic responses. New methods for earlier, more reliable and cheaper diagnosis would be one of the most effective means to reduce the cost load and achieve better cell-selectivity and delivery efficiency of antitumor agents.

### SUMMARY

Magnetic-field responsive nanowires can be functionalized with antibodies to target and manipulate biological cells. As an example, nanowires bound to antibodies can be applied to kill cancer cells. Once attached to the cells, an alternating magnetic field can be applied by external magnets and due to the magnetic-field responsiveness of the nanowires, they will move or vibrate, thereby transmitting energy to the targeted cells, which can induce apoptosis or other cell-disruptive events.

In one aspect, a composition includes a plurality of nanowires, at least a portion of the plurality of nanowires being responsive to a magnetic field and a plurality of targeting moieties, each of the moieties having a first affinity for a surface of a predetermined cell type and a second affinity for a nanowire in the plurality of nanowires, wherein at least one moiety in the plurality of targeting moieties is in contact with a nanowire of the plurality of nanowires, wherein the plurality of nanowires responds to a magnetic field.

In certain embodiments, the first affinity can include the targeting moiety configured to have specific recognition and binding to the surface of the predetermined cell type. For example, the antibody can have a first affinity to a membrane receptor of a cancer cell. The second affinity can include the targeting moiety is configured to conjugate to the nanowire through the chemical groups of the moiety with the surface of the nanowire. The moieties can be randomly distributed or a patterned distribution on a nanowire in the plurality of nanowires.

In certain embodiments, the targeting moiety can be an antibody. The antibody can have a second affinity for a nanowire in the plurality of nanowires wherein the antibody is a chemically modified antibody. The chemically modified antibody can include sulfhydryl groups.

In certain embodiments, each of the plurality of nanowires can include nickel, cobalt, iron, or alloys or combinations thereof. Each nanowire in the plurality of nanowires can include a gold coating. At least a portion of the plurality of nanowires can be magnetic or magnetostrictive.

In another aspect, a method of making a composition can include forming a plurality of nanowires wherein the diameter and length of each nanowire are controllable, configuring the plurality of nanowires to be biocompatible, and contacting a plurality of targeting moieties with a plurality of nanowires, wherein the moieties have a first affinity for a surface of a predetermined cell type and a second affinity for a nanowire in the plurality of nanowires, wherein the plurality of nanowires responds to a magnetic field whereby a force is exerted on the predetermined cell type causing cell modification. The method can include forming a plurality of nanowires wherein the diameter and length of each nanowire are predetermined before contacting a plurality of targeting moieties with a plurality of nanowires, wherein the moieties have a first affinity for a surface of a predetermined cell type and a second affinity for a nanowire in the plurality of nanowires, wherein the plurality of nanowires responds to a magnetic field whereby a force is exerted on the predetermined cell type causing cell modification.

In certain circumstances, the method can include configuring the plurality of nanowires to be biocompatible before contacting a plurality of targeting moieties with a plurality of nanowires, wherein the moieties have a first affinity for a surface of a predetermined cell type and a second affinity for a nanowire in the plurality of nanowires, wherein the plurality of nanowires responds to a magnetic field whereby a force is exerted on the predetermined cell type causing cell modification.

In certain embodiments, the method can include forming the plurality of nanowires includes electrodeposition into nanopores of a membrane. The length of each nanowire in the plurality of nanowires can be dependent on electrodeposition time. The diameter of each nanowire in the plurality of nanowires can be dependent on the nanopore size of the membrane. The membrane can be an alumina membrane, which can be formed by an anodization process including a highly pure aluminum substrate. The alumina membrane can have a honeycomb structure.

In certain circumstances, the method can include coating the plurality of nanowires with gold, for example, by electroless deposition of gold into nanopores of a membrane.

In another aspect, a method of cell modification can include administering a composition including a plurality of nanowires, at least a portion of the plurality of nanowires being responsive to a magnetic field, applying the magnetic field to the composition, and modifying cell of the predetermined cell type, wherein a plurality of targeting moieties, each of the moieties having a first affinity for a surface of a predetermined cell type and a second affinity for a nanowire in the plurality of nanowires, wherein the at least one moiety in the plurality of moieties is in contact with a nanowire of the plurality of nanowires, wherein the plurality of nanowires responds to a magnetic field whereby a force is exerted on the predetermined cell type causing cell modification. Applying the magnetic field can include subjecting the plurality of nanowires to a magnetic field strength effective to cause the plurality of nanowires to vibrate. The method can include introducing the composition to the patient. The magnetic field can be applied inside or external to the patient.

Other aspects, embodiments, and features will be apparent from the folio description, the drawings, and the claims.

### BRIEF DESCRIPTION OF THE DRAWINGS

FIG. 1A is a schematic of a biofunctionalized magnetic nanowire conjugated to a targeting moiety, specifically, at least one antibody. FIG. 1B is a schematic of biofunctionalized magnetic nanowires used for cell modification, specifically antibody-based cancer therapy.

FIG. 2 is a schematic depicting the dimensional change observed due to the alignment of magnetic moments under the presence of a magnetic field H, wherein arrows represent the magnetic moments.

FIG. 3 is a schematic depicting the step by step nanowire fabrication process, wherein (a) is the aluminum substrate; (b) shows the first anodization; (c) shows the aluminum template; (d) shows the second anodization; (e) shows the widening and thinning the alumina nanomembrane; and (f) shows the magnetic metal filling.

FIG. 4 is a graph depicting Hysteresis loops for an array of NiCo nanowires (average diameter=35 nm, length=1 μm) with the applied field H parallel (outer lines at center) and perpendicular (inner lines at center) to the wires' longitudinal axis.

FIG. 5 is a schematic depicting a normal vasculature versus a leaky one, which is a typical feature of tumors.

FIG. 6 is a schematic depicting an anodization cell wherein Uₐₚ is the applied voltage, the gray solution represents the oxalic acid, and the hole diameter of the cell=16 mm.

FIG. 7 is a graph depicting the electrodeposition voltage/current-time profile.

FIG. 8 is a schematic depicting the electrochemical deposition of gold in PAA membranes.

FIG. 9 is a schematic depicting the antibody modification process.

FIG. 10 is a schematic depicting the one color microarray experimental workflow.

FIG. 11 is a photograph of a 96-well microtiter plate after an MTT assay wherein increasing amounts of cells resulted in increased (darker) coloring.

FIG. 12 shows photographs depicting the experimental setup for the application of an alternating magnetic field to cultured cancer cells. (a) Experimental setup described in Abu Samra et al 2011; and (b) Modifications on setup (a).

FIG. 13 shows SEM images of honeycomb alumina nanomembranes. (a) PD=35 nm; ID=100 nm. Each pore contains a Co nanowire; (b) PD=55 nm. Each pore contains a Ni nanowire.

FIG. 14 shows SEM images of fully (a) and partially released (b,c) NiCo nanowires. (a) Diameter=35 nm, length=1 μm, 1 h NaOH; (b,c) Diameter=55 nm, length=2 μm, (b) 2 min NaOH followed by CPD (critical point drying) and (c) extra 2 min NaOH followed by CPD.

FIG. 15 shows SEM images of Ni nanowires (diameter=35 nm, length=1 mm) after 4 min NaOH+CPD+Au sputtering. Top (a) and 30° tilted (b-c) views.

FIG. 16 shows optical images of SKOV3 (ovarian cancer cell line) at different time points and different nanowires concentration.

FIG. 17 shows charts depicting gene ontology terms that were upregulated when a concentration of 1000 CoNi nanowires per cell were added to ovarian cancer cells in culture.

FIG. 18 shows representative SEM pictures of magnetic nanowires pre-treated before gold sputtering.

### DETAILED DESCRIPTION

Magnetic micro- and nanomaterials are becoming increasingly interesting for biomedical applications that include drug delivery, hyperthermia treatment, bioseparation and contrast-enhancement in magnetic resonance imaging. See, for example, Kubo, T. et al., 2000. International Journal of Oncology, 17(2), pp. 309-315; Jurgons, R. et al., 2006. Journal of Physics: Condensed Matter, 18(38), p. S2893-S2902; Hergt, et al. 2008. Journal of Physics: Condensed Matter, 20(38), p. 385214; Hilger, et al. 2005. IEEE Proceeding Nanobiotechnology, 152(1), pp. 33-9; Hultgren, A. et al., 2003. Journal of Applied Physics, 93(10), pp. 7554-7; and Weissleder, R. et al., 1997. Journal of magnetic resonance imaging, 7(1), pp. 258-63, each of which has been incorporated by reference in its entirety.

Magnetic nanostructures offer important advantages in comparison with classical cancer treatments and non-magnetic nanoparticles. Firstly, due to their magnetic nature, they can be activated, controlled and manipulated remotely with a magnetic field. Additionally, their size can be controlled within a range extending from a few nanometers in size to hundreds of nanometers in size, putting them in the same size range as many biological entities like enzymes, cell receptors, genes, bacteria, etc, See, for example, Giouroudi, I. & Kosel, J., 2010. Recent patents on nanotechnology, 4(2), pp. 111-8, which is incorporated by reference in its entirety.

However, magnetic nanoparticles do not interact a controlled manner with biological systems unless they are biocompatible and biofunctionalized. Biocompatibility refers to “the ability of a material to perform with an appropriate host response in a specific application,” while biofunctionalization is the chemical surface modification needed for nanoparticles to become bioactive when encountering biologic entities, i.e. magnetic nanoparticles can be functionalized with antibodies that target specific cells lines. See, for example, Williams, D. F., 1987. Definitions in biomaterials. In Proceedings of a Consensus Conference of the European Society for Biomaterials. N.Y.: Elsevier, which is incorporated by reference in its entirety.

The main goals of conventional cancer therapies (e.g., drugs or radiation) are DNA damage and prevention of DNA synthesis to stop cell replication. These goals can be achieved with magnetic nanoparticles by inducing hyperthermia; delivering drugs or by transmitting magnetomechanical forces. See, for example, Ophardt, C. E., 2003. Virtual ChemBook., Elmhurst College; Hergt, et al, 2008. Journal of Physics: Condensed Matter, 20(38), p. 385214; Hilger, et al. 2005. IEEE Proceedings—Nanobiotechnology, 152(1), pp. 33-9; Dutz, S et al., 2007. International Federation for Medical and Biological Engineering Proceeding. pp. 271-274; Jurgons, R. et al., 2006. Journal of Physics: Condensed Matter, 18(38), p. 52893-52902; McBain, et al. 2008, International journal of Nanomedicine, 3(2), pp. 169-80; Veiseh, et al. 2010. Advanced Drug Delivery Reviews, 62(3), pp. 284-304; Kim, Dong-Hyun et al., 2010. Nature Materials, 9 (November 2009), pp. 165-71, each of which is incorporated by reference in its entirety.

Magnetic nanoparticles are able to absorb energy from an alternating magnetic field and re-emit that energy as heat. Magnetic hyperthermia involves placing magnetic particles in or near a tumor, subjecting the particles to an alternating magnetic field to increase local temperature, thereby causing necrosis of the tumor cells. In delivering this current experimental cancer treatment, relevant factors to consider include the killing temperature, which also depends on a repertoire of factors such as the size and composition of the magnetic nanoparticles used, and the values of amplitude and frequency of the alternating magnetic field. See, for example, Hergt, et al. 2008. Journal of Physics: Condensed Matter, 20(38), p. 385214, which is incorporated by reference in its entirety. The use of nanoparticles functionalized with different therapeutic payloads (chemical, radiological or biological) began more than 30 years ago and is not exclusive of magnetic nanoparticles. The use of magnetic nanoparticles offers advantages like guidance of the therapeutic payload (i.e., a chemical drug formulation, a radionucleotide or a gene) using a magnetic field and visualization using magnetic resonance imaging, of the particles after the therapeutic payload is delivered. See, for example, Jurgons, R. et al., 2006. Journal of Physics: Condensed Matter, 18(38), p. S2893-S2902, which is incorporated by reference in its entirety. When a magnetic field is applied on magnetic microstructures (such as gold coated permalloy microdisks) attached to cancer cells, a magnetomechanical force is transmitted to the cells. See, for example, Rozhkova, E. a. et al., 2009. Journal of Applied Physics, 105(7), pp. 105-7 and Kim, Dong-Hyun et al., 2010, Nature Materials, 9 (November 2009), pp. 165-71, each of which is incorporated by reference in its entirety.

Functionalization methods can work by themselves, more than one at the same time, or as an enhancement of a conventional therapy. For example, a network of silica was loaded with iron oxide superparamagnetic nanocrystals and with immobilized single stranded DNA onto its surface. Additionally, the complementary DNA sequence was attached to magnetic nanoparticles. These nanoparticles were able to cap the pores of the magnetic silica network upon hybridization of both DNA strands, and, because heat was produced when an alternating magnetic field was applied, the DNA linkage was reversible resulting in an “on-off” release mechanism. See, for example, Ruiz-Hernández, E., Baeza, A. & Vallet-Regi, M., 2011. Smart drug delivery through DNA/magnetic nanoparticle gates. American Chemical Society nano, 5(2), pp. 1259-66, which has been incorporated by reference in its entirety. The cancer treatment involved magnetic nanoparticles in which both hyperthermia and drug delivery approaches were used.

Applying a magnetic field actuates the nanowires. For example, the magnetic field applied to magnetostrictive nanowires can cause the nanowires to extend and contract (e.g., oscillation or vibration). The magnetic field can be constant or varying; one example of a varying magnetic field is a regularly oscillating magnetic field. By properly designing the nanowire lengths and diameters, the resonant frequency (of an oscillating field) at which each type of nanowire responds to the magnetic field can be altered.

The tunability of the physical properties of the nanowires makes them uniquely suited for creating therapies aimed at cell disruption. Instead of involving both hyperthermia and drug delivery approaches as had been done previously, the application of magnetic nanowires, allow for exertion of mechanical force on a cell, inducing apoptosis. Nanowires functionalized with antibodies were developed, with the antibodies being specific for cancer cells. In this way, when the nanowires are subjected to an appropriate magnetic field to induce vibration, the vibration is transmitted to the cell via the antibody. This transmits a mechanical force to the targeted cells, which is expected to induce apoptosis of the cells.

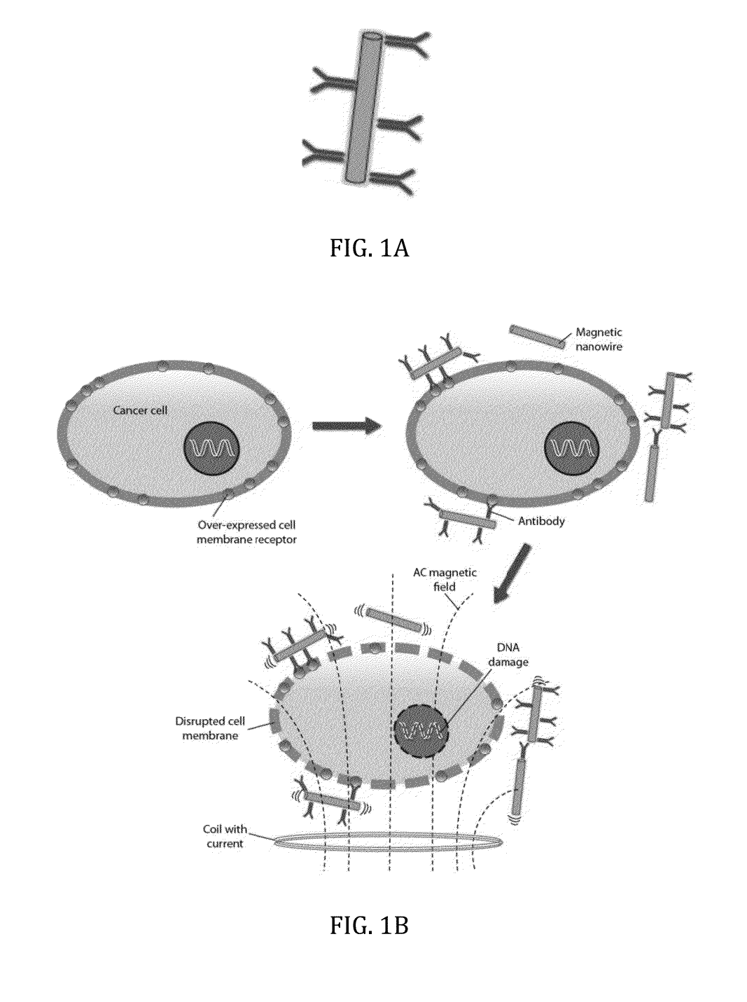

As depicted in FIG. 1 for cells, for example, cancer cells, the magnetic nanowire is bound to antibodies that specifically bind to the target cells. FIG. 1A shows the magnetic nanowire in contact with at least one targeting moiety, for example, an antibody. FIG. 1B shows the method of cell modification with nanowires bound to targeting moieties, for example, the inducement of apoptosis in cancer cells with nanowires bound to antibodies. The magnetic nanowires must first be made biocompatible, which can be accomplished in many ways, one of which being coating the nanowires with gold. The biocompatible nanowires can then be biofunctionalized. For example, the biofunctionality can involve binding antibodies of a particular specificity to the nanowires. At least one antibody can be bound to a nanowire. The biofunctionalized nanowires can then bind to the target cells via the bound antibodies.

When a magnetic field is applied, for example, an AC magnetic field, the magnetostrictive nature of the nanowires causes the nanowires to elongate and shrink, or vibrate. The vibration transmits a force to the targeted cells, for example, cancer cells. The force can result in apoptosis of the target cell.

Nanowires and nanorods are straight solid one-dimensional high aspect ratio (length divided by width) nanomaterials. A nanowire usually has a higher aspect ratio than a nanorod. Nanowires generally include at least one substantially crystalline or amorphous material. In certain embodiments, the nanostructure can include a core of a first material and at least one shell of a second (or third etc.) material, where the different material types are distributed radially about the long axis of a nanowire, a long axis of an arm of a branched nanowire, or the center of a nanocrystal, for example. Depending on the application and other design parameters, nanowires can have an aspect ratio (length-to-width ratio) of for example, 1, 5, 10, 100, 250, 500, 800, 1000 or higher. A nanowire preferably has a diameter of less than approximately 500 nm at its maximum point, and the diameter along the longitudinal axis preferably varies by less than approximately 10% over the section exhibiting the maximum change in diameter. Nanowires structures with a diameter in the range of approximately 5 nm to approximately 500 nm, with lengths in the range of approximately 10 nm to approximately 100 μm, can be formed.

Nanowires can be produced by a number of methods including electrodeposition, chemical etching, vapor-liquid-solid (VLS) synthesis, or solution-phase synthesis, to name a few examples. The nanowire can be substantially crystalline or amorphous, and can include semiconductors, oxides, or other materials.

Nanowires can have various cross-sectional shapes, including, but not limited to, circular, square, rectangular and hexagonal. In each case, the term “diameter” is intended to refer to the effective diameter, as defined by the average of the major and minor axis of the cross-section of the structure. Nanowires display several size-dependent properties, including electronic, optical and magnetic properties. Magnetic nanowires can be made of pure metals including rare earth metals or alloys, consisting of, e.g., cobalt, nickel, iron, gallium, terbium, dysprosium and combinations thereof. The use of different metals or of alloys allows for the tailoring of the magnetic responses under applied magnetic fields.

In some examples, nanowires can have a generally cylindrical in shape with a diameter in the range from 5 to 500 nm and length up to 100 μm. See, for example, Varadan, V, Chen, L. & Xie, J., 2008. Nanamedicine, John Wiley & Sons, ed., Sussex, United Kingdom, which has been incorporated by reference in its entirety. Recently, nanowires were added to cells in culture looking for a large repertoire of applications. The manner in which cells in culture behave when magnetic nanowires are added mainly depends on the material, aspect ratio and concentration of the nanowires as well as the cell type and total cell culture time with the nanowires.

In one example showing biological uses of nanowires, mouse fibroblasts were manipulated using Ni nanowires added to the cells in culture. The cells had affinity for the wires that remained attached to the cells even after adding trypsin-EDTA to detach them from the culture plate. After the cells were detached from the plate, a magnetic field gradient was applied, efficiently separating the portion of cells that had internalized the nanowires. Thus, nanowires are able to exert forces on cells. See, for example, Hultgren, A. et al., 2003. Journal of Applied Physics, 93(10), pp. 7554-7, which is incorporated by reference in its entirety. In a related study, the cells were found to bind to the nanowires through integrin receptors, as indicated by the formation of focal adhesions along the length of the nanowires. Cell separation was optimized when the length of the nanowires matched the average diameter of the cells in suspension. The chemotherapeutic agent mitomycin-C was used to make the same cell line larger in diameter. The corresponding nanowires for which the length matched the new cell diameter were the ones that more efficiently separate the cells. See, for example, Hultgren, A. et al., 2005. Optimization of yield in magnetic cell separations using nickel nanowires of different lengths. Biotechnology progress, 21(2), pp. 509-15, which has been incorporated by reference in its entirety.

Ni nanowires have been used on the same cell line where the goal was to kill the cells when an external magnetic field was applied (Ring et al. 2008). Cells easily internalized the wires, and despite the reported moderate cytotoxic effects of nickel, there was almost no IL-6 upregulation after 12 h of culture. That result indicated that these nanowires have potential as biocompatible therapeutic materials. See, for example Ring, A. O. et al., 2008. Induction of Cell Death by Magnetic Actuation of Nickel Nanowires Internalized by Fibroblasts. The Journal of Physical Chemistry C Letters, 112, pp. 15085-15088, which has been incorporated by reference in its entirety. Cells with wires showed no viability change in the absence of a magnetic field. However, when a rotating magnetic field of 120 mT was applied for 20 minutes, cell viability dropped almost 90%. See, for example, Schmalz, G., Schuster, U. & Schweikl, H., 1998. Influence of metals on IL-6 release in vitro. Biomaterials, 19(18), pp. 1689-94, which has been incorporated by reference in its entirety.

Later, the same cell line was studied but this time the material and fabrication process of the wires were fabricated by a controlled assembly of iron oxide nanoparticles (Safi et al. 2011). As before, the wires were internalized after 24 h by the cells and remained in membrane bound compartments or in the cytosol. Interestingly, the cells were able to degrade the wires in a short time period (days) and seen in Table 1. See, for example, Safi, M. et al., 2011. ACS Nano, 5(7), pp. 5354-5364, which has been incorporated by reference in its entirety.

TABLE 1
Overview of research involving addition of
magnetic nanowires to cells in culture.

| | | | Nanowires to | |
|---|---|---|---|---|
| | Nanowires | Nanowires | cells | |
| Reference | material | dimensions | concentration | Highlights |
| Hultgren | Nickel | D = 350 nm | <1:3 | Nanowires |
| et al. 2003 | | L = 35 μm | | were always |
| Hultgren | | D = 350 nm | 1:5 to 1:20 | internalized |
| et al. 2005 | | L = 5-35 μm | | by NIH-3T3 |
| Fung et al. | | D = 198-280 nm | Not | cell line |
| 2008 | | L = 3-6 μm | mentioned | (mouse |
| Safi et al. | Iron | D = 200 nm | 30:1 | fibroblast) |
| 2011 | oxide | L = 1-40 μm | | |

D = diameter; L = length.

Although the cellular uptake of magnetic nanostructures has not been demonstrated, gold nanospheres and nanowires of different sizes are taken up by HeLa (human cervical cancer) cells, See, for example, Chithrani, B. D., Ghazani, A. a & Chan, W. C. W., 2006. Determining the size and shape dependence of gold nanoparticle uptake into mammalian cells. Nano letters, 6(4), pp. 662-8, which has been incorporated by reference in its entirety. Nanospheres of 14, 30, 50, 74, and 100 nm diameter as well as wires of 40×14 nm and 74×14 nm were tested, and it was found that the uptake of these nanostructures is highly dependent on their size and shape. For gold nanospheres, the highest uptake occurred for structures of 50 mm diameter, which is in agreement with a reported upper size limit of endocytic vesicles of 100 nm. See, for example, Osaki, F. et al., 2004. A quantum dot conjugated sugar ball and its cellular uptake. On the size effects of endocytosis in the subviral region. Journal of the American Chemical Society, 126(21), pp. 6520-1, which has been incorporated by reference in its entirety. However, it is unclear why particles smaller than 50 nm were not internalized by the cells. Concerning nanowires, their uptake was lower than spherical nanoparticles. Cellular uptake of nanowires with lower aspect ratio (1:3) was greater than higher aspect ratio (1:5) ones.

The structure and shape of magnetic domains (mainly longitudinal or circular in case of wires) defined by the anisotropies (shape, crystalline, elastic) of a material. As an example, in Co nanowire arrays, the crystalline term overcomes shape anisotropy resulting in a circular magnetization. See, for example, Manuel Vazquez 2012, Hernández-Vélez, M., 2006. Nanowires and 1D arrays fabrication: An overview. Thin Solid Films, 495(1-2), pp. 51-63, which has been incorporated by reference in its entirety.

For example, shape anisotropy of Ni nanowire arrays overcomes magnetocrystalline anisotropy making the parallel hysteresis loop square-shaped. See, for example, Vázquez, M. et al., 2005. Journal of Magnetism and Magnetic Materials, 294(2), pp. 174-181, which is incorporated by reference in its entirety. Contrary, no squareness is seen in neither hysteresis loops for Co nanowires, at least for large diameter wires (100 nm and above). For them, crystalline anisotropy has a stronger effect. See, for example, Ferré, R. & Ounadjela, K., 1997. Physical Review B, 56(21), pp. 66-75, which is incorporated by reference in its entirety.

Magnetic nanowires can be manipulated in various ways using magnetic fields. An inhomogeneous magnetic field exerts a translational force to magnetic nanowires that can be used to move or confine them. If the magnetization of the nanowires is not aligned with the direction of the applied field, a torque will be exerted on the wires causing them to align in a DC field or oscillate in a low-frequency AC field.

Magnetostriction is a property of ferromagnetic materials such as iron (Fe), nickel (Ni) and cobalt (Co). When placed in a magnetic field, these materials change size and/or shape. See, for example, Magnetostrictive Linear Position Sensors. MTS Systems Corp, USA, which is incorporated by reference in its entirety.

In 1842, James Joule noted that a ferromagnetic sample changed its length on the presence of a magnetic field. This effect of change in size because of a magnetic force (striction=compression) was noted as magnetostriction and named after him as Joule effect (Bhattacharya 2005).

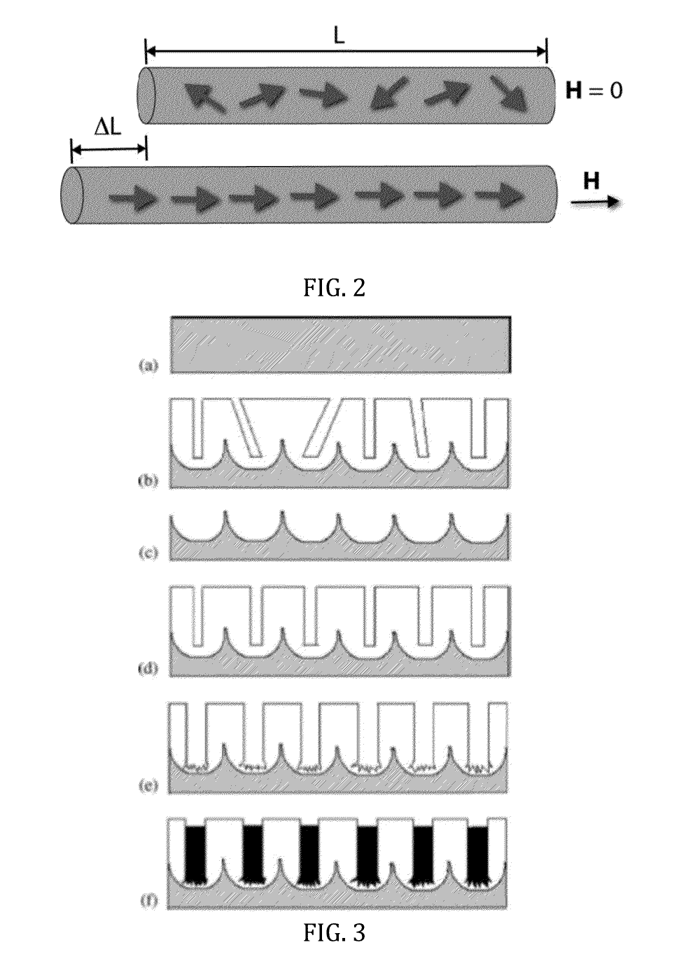

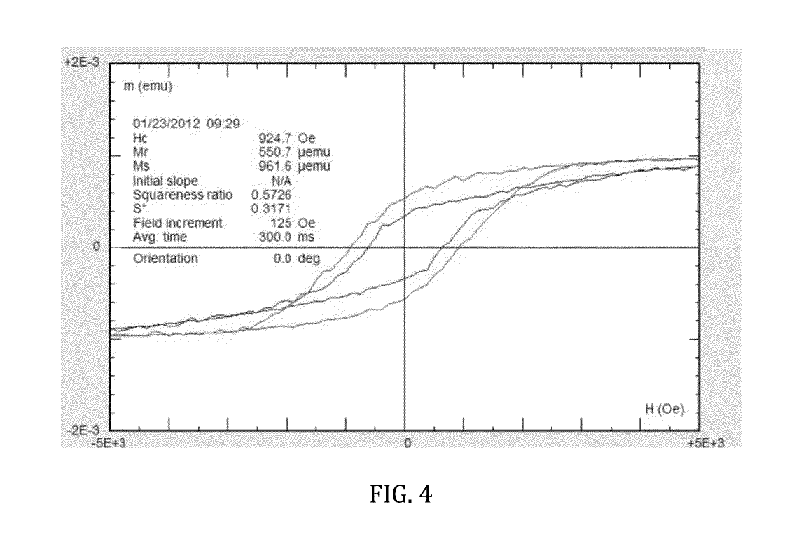

This phenomenon occurs because of the re-orientation followed by an alignment of the magnetic domains of the material when an external field is applied leading to a change in length in the case of magnetic wires as seen in FIG. 2. A typical example of magnetostriction is the familiar “electric hum” in AC electrical devices such as transformers. See, for example, Carl R, N. Georgia S. U., 2001. Hyperphysics—Magnetostriction. Available at: http://hyperphysicsphy-astr.gsu.edu/hbase/solids/magstrict.html [Accessed Sep. 26, 2011], which has been incorporated by reference in its entirety.

In formal treatments, the magnetostrictive coefficient Λ of a magnetic material is proportional to its fractional change in length as the magnetization (M) increases from zero to its saturation value (Mₛ)

$$Λ ∝ \frac{Δ L}{L} = \frac{{L}_{{M}_{s}} - {L}_{M = 0}}{L} \qquad ( 1 )$$

Λ can be positive (Λ>0 [symbol] L_{M}_{ₛ}>L_{M=0}, elongation) or negative (Λ<0 [symbol] L_{M}_{ₛ}<L_{M=0}, shrinkage) and changes with temperature. Table 2 shows the values of Λ for some ferromagnetic materials at room temperature. The last three metals shown have the highest magnetostriction because of the magnetic anisotropy of rare earth metals like terbium (Tb) and dysprosium (Dy). See, for example, Bhattacharya, B., 2005. Directions magazine (Indian Institute of Technology Kanpur), 7(2), pp. 35-40, which is incorporated by reference in its entirety.

TABLE 2
Magnetostriction coefficient value for the
most common ferromagnetic materials.

| Material | Λ (×10⁻⁵) |
|---|---|
| Cobalt (Co) | −5-6^{1,2} |
| Iron (Fe) | 0.8-1.4^{1,2} |
| Nickel (Ni) | −2.5-4.7^{1,2} |
| Permalloy^{(a)} | 2.7² |
| Galfenol^{(b)} | 20-40³ |
| DyFe₂ | 65² |
| Terfenol-D^{(c)} | 80-240^{2,3} |
| TbFe₂ | 263² |

¹(Brown 1958);
²(Bhattacharya 2005);
³(Etrema_Products_Inc. 2003)
^{(a)}Alloy consisting in 80%Ni—20%Fe;
^{(b)}Alloy of gallium (Ga) and Fe:
^{(c)}Alloy named after terbium, iron (Fe), Naval Ordnance Laboratory (NOL), and the D comes from dysprosium.

The most common fabrication process for magnetic nanowires involves electroplating the material into the nanopores of an insulating membrane, which requires a conductive substrate at the bottom. Besides sophisticated techniques to fabricate a nano membrane such as electron-beam, X-ray or nano-imprint lithography an alternative simple and inexpensive method is anodization to create porous anodic alumina (PAA) as a substrate. PAA is among the most widely used self-ordered nanostructured materials because it is possible to obtain pores with highly controlled diameter sizes from tens to hundreds of nanometers that self-arrange during anodization into hexagonal symmetry. See, for example, Masuda, H. & Fukuda, K., 1995. Science, 268(5216), pp. 1466-8, and Nielsch, K. et al., 2000. Advanced Materials, 12(8), pp. 582-586, each of which is incorporated by referenced in its entirety.

A two-step anodization process using highly pure aluminum as a substrate creates the PAA. After that, the nanowires are formed by electroplating the desired metal (such as Ni, Go, Fe and FeNi) into the pores of the PAA. See, for example, Masuda, H. & Fukuda, K., 1995. Science, 268(5216), pp. 1466-8; Nielsch, K. et al., 2000. Advanced Materials, 12(8), pp. 582-586; Nielsch, K. et al., 2001. Applied Physics Letters, 79(9), p. 1360; Vázquez, M. et al., 2004. Journal of Applied Physics, 95(11), p. 6642; and Vázquez, M. et al., 2005. Journal of Magnetism and Magnetic Materials, 294(2), pp. 174-181, each of which is incorporated by referenced in its entirety. FIG. 3 shows step by step the fabrication process. See, for example, Pirota, K et al., 2004. Journal of Alloys and Compounds, 369(1-2), pp. 18-26, which is incorporated by reference in its entirety.

Anodization involves immersing a metal in an electrolytic bath (i.e. oxalic acid, sulfuric acid or phosphoric acid) at a fixed temperature and voltage. A chemical reaction will lead to the formation of a porous alumina layer on top of the aluminum substrate.

During the first anodization (FIG. 3b), high aspect ratio non-parallel pores are formed in a hexagonal array. This process is followed by a complete dissolution of the alumina membrane by a wet chemical etching process (FIG. 3c), leaving on the aluminum substrate regular hexagonal indentations where the pore formation will preferably start during a second anodization. After the second anodization straight pores are obtained (FIG. 3d). Anodization times have different effects on the nanowire fabrication process. The first anodization time will determine the size of the nanopore hexagonal domains and the homogeneity of the nanopores diameter. Particularly, for a first anodization time of 72 h Vázquez et al. 2005 found an increase in size of the hexagonal domains with a reduction in homogeneity of the pore diameter of about 6% in comparison with the array obtained after only 3 h. See, for example, Vazquez, M. et al., 2005. Journal of Magnetism and Magnetic Materials, 294(2), pp. 174-1.81, which is incorporated by reference in its entirety. A suitable first anodization time is 24 h because, at the end, alumina pores are parallel with a homogeneous pore diameter. See, for example, Pirota, Kleber, 2011. Group optical and magnetic properties of solids. University of Campinas, which is incorporated by reference in its entirety.

The electrolyte and voltage used for the first anodization process determined the pore diameter and interpore distance (Table 3).

TABLE 3
Anodization parameters. (Vázquez et al 2005)

| | Voltage | Interpore distance | Pore diameter |
|---|---|---|---|
| Electrolytic bath | (V) | (nm) | (nm) |
| Sulfuric acid | 25 | 65 | 25 |
| Oxalic acid | 40 | 105 | 35 |
| Phosphoric acid | 195 | 500 | 180 |

The second anodization time determined the length of the pores. See Table 4.

TABLE 4
Pore growth rates during 2^{nd} anodization.

| | Electrolytic | Voltage | Temp. | Pore growth |
|---|---|---|---|---|
| Reference | bath | (V) | (° C.) | rate |
| Nielsch 2000 | 0.3M oxalic acid | 40 | 2 | 1 μm/h |
| K. R. Pirota | 0.3M oxalic acid | 40 | 4 | 2 μm/h |
| 2011 | | | | |

Once the alumina membrane is anodized, and before proceeding to the final step that is electroplating, the alumina membrane needs to be thinned (creation of dendrites in the bottom of the pores, FIG. 3e) to create electrical contact between the electroplating solution and the aluminum (alumina is an insulator).

After thinning the alumina the sample is ready for electroplating. However, an additional (optional) step might be useful before proceeding. A pore widening process using wet chemical etching can be done if wires with larger diameters than the ones listed in Table 3 are needed for a specific application. When using 0.3M oxalic acid at 30° C., equation 2 determines the final pore diameter (PD)

$$PD = {PD}_{initial} + 10 \frac{nm}{h} \times t \qquad ( 2 )$$

After the alumina thinning and the optional pore-widening step, the sample is ready for electroplating. Electroplating consists of applying a voltage to an electrolyte containing the metal salts of the material of interest (i.e. Co or Ni). The positive ions of Ni or Co are attracted to the aluminum substrate and deposited there. Some common electroplating recipes are shown in Table 5.

TABLE 5
Ni and Co electroplating solution recipes.

| | | Temp. | |
|---|---|---|---|
| Material | Recipe | (° C.) | References |
| Ni | 300 g/L NiSO₄•6H₂O | 35 | Nielsch 2000, |
| | 45 g/L NiCl₂•6H₂O | | Pirota 2004 |
| | 45 g/L H₃BO₃ | 40 | Pirota 2011 |
| Co | 300 g/L CoSO₄•7H₂O | 40 | Pirota 2011 |
| | 45 g/L CoCl₂•6H₂O | | |
| | 45 g/L H₃BO₃ | | |

There are two main methods of electroplating to obtain completely and uniformly filled pores. See, for example, Nielsch. K. et al., 2001. Applied Physics Letters, 79(9), p. 1360, which is incorporated by reference in its entirety. The first method uses direct current. The alumina membrane needs to be detached from the aluminum substrate by a chemical etching process. Afterwards, a metallic contact (i.e. gold) is sputtered on one of the sides of the freestanding alumina membrane. This method is only applicable for thick freestanding alumina membranes (>20 μm) that are stable enough for handling. For nanofabrication purposes 20 μm alumina thickness implies very long anodization times and other complications.

In the second method, the aluminum remains attached to the alumina membrane and a pulsed voltage-current electrodeposition profile is applied. The reason of using pulsed electrodeposition is to obtain a more homogenous pore filling, keeping the ion distribution of the solution even. See, for example, Pirota, K, 2011 Group optical and magnetic properties of solids, Pinto Leitao, D. C., 2010. Micro and Nano Patterned Magnetic Structures. University of Porto and Vázquez, M. & Vivas, L., 2011. Magnetization reversal in Co-base nanowire arrays. Physica Status Solidi (B), 248(10), pp. 2368-2381, each of which has been incorporated by reference in its entirety. Also, using pulsed electrodeposition gives a better deposition control of the metal and prevents damage to the alumina membrane helping in rebuilding the pores.

Many authors have characterized arrays of magnetic nanowires. Some of them have characterized Ni arrays, Co arrays, compared Ni and Co arrays magnetic behavior, and tertiary FeCoNi arrays. See, for example, Nielsch, K. et al., 2001. Applied Physics Letters, 79(9), p. 1360; Pirota, K et al., 2004. Journal of Alloys and Compounds, 369(1-2), pp. 18-26; Sun, L. et al., 2005. IBM Journal of Research and Development, 49(1), pp. 79-102; Vázquez, M. et al., 2005. Journal of Magnetism and Magnetic Materials, 294(2), pp. 1.74-181; Silva, E. et al., 2006. Physica B: Condensed Matter, 384(1-2), pp. 22-24; Ferré, R. & Ounadjela, K., 1997. Physical Review B, 56(21), pp. 66-75; and Sharma, G. & Grimes, C. a., 2004. Journal of Materials Research, 19(12), pp. 3695-703, each of which is incorporated by reference in its entirety.

A characterization work on arrays of Ni nanowires concluded that crystal anisotropy is small compared to the shape anisotropy and the magnetization lies along the wire axis. Seem for example, Ferré, R. & Ounadjela, K., 1997, Physical Review B, 56(21), pp. 66-75, which is incorporated by reference in its entirety. When increasing the length of the wires, larger values for both H_{c} and Mᵣ were measured on the parallel hysteresis loop. See, for example, Vázquez, M. et al., 2005. Journal of Magnetism and Magnetic Materials, 294(2), pp. 174-181 and Nielsch, K. et al., 2001. Applied Physics Letters, 79(9), p. 1360, each of which is incorporated by reference in its entirety. Moreover, when the nanowire diameter was decreased, the same behavior of H_{c} and Mᵣ was observed for the parallel hysteresis loop.

in the case of Co nanowire arrays with larger diameters (100 nm and above) none of the hysteresis loops (neither parallel nor perpendicular) is square-shaped. They present a strong crystal anisotropy with an easy axis oriented nearly perpendicular to the axis of the wire. See, for example, Ferré, R. & Ounadjela, K., 1997. Physical Review B, 56(21), pp. 66-75, which is incorporated by reference in its entirety. For small diameter ones (35 nm according) the shape of the parallel hysteresis loops is square-shaped as the one of the Ni nanowires.

FeCoNi ternary alloy nanowire arrays with 32-106 nm in diameter and 8475 nm length showed a magnetization easy-axis along the length of the wire. Squareness ratio decreased as the nanowire diameter increases and coercivity was found to increase as the aspect ratio of the wire increases. See, for example, Sharma, G. & Grimes, C. a., 2004. Journal of Materials Research, 19(12), pp. 3695-703, which is incorporated by reference in its entirety.

Most biomedical applications use superparamagnetic particles. See, for example, Sharma, G. & Grimes, C. a., 2004. Journal of Materials Research, 19(12), pp. 3695-703, which is incorporated by reference in its entirety. However, some researchers prefer using different kinds of iron oxides (ferrimagnets) such as Fe₃O₄ (magnetite) and γ-Fe₂O₃ (maghemite) that have been proven to be well tolerated by the human body (R Hergt et al. 2006). A different approach consists in coating ferromagnetic materials like nickel (Ni), cobalt (Co), iron (Fe) or permalloy (80% Ni-20% Fe alloy) with a thin layer of gold, a biocompatible metal. See, for example, Shukla, R., 2007. National Centre for Cell Science and Anon, 2003. Materials World Magazine, pp. 12-14, each of which is incorporated by reference in its entirety.

Cytotoxicity of magnetic materials has been addressed by different researchers. Ni nanowires to mouse fibroblasts cells in culture and measured cell viability with a MTT assay. See, for example, Fung, A. O. et al., 2008. Induction of Cell Death by Magnetic Actuation of Nickel Nanowires Internalized by Fibroblasts. The Journal of Physical Chemistry C Letters, 112, pp. 15085-15088, which has been incorporated by reference in its entirety. It was found that cell viability was not significantly affected by the nanowires at 12 hours.

When adding Ni nanowires to a human leukemia cell line (THP-1), the cells remained viable for 24 h with no significant decrease in cell count for a concentration of 10 nanowires plated to every cell. See, for example, Byrne, F. et al., 2009. Journal of Magnetism and Magnetic Materials, 321(10), pp. 1341-5, which is incorporated by reference in its entirety. Moreover, THP-1 cells remained viable for 10 h periods even after ingesting surface-oxidized Ni wires at concentrations up to 100 nanowires per cell in the culture media. However, the cytotoxicity effects were notable when the concentrations increased up to 500 nanowires per cell.

Fe and iron oxide (γ-Fe₂O₃) nanowires have also been tested on different cell lines. Cytotoxicity and cellular uptake of Fe nanowires by a human cervical cancer cell line (HeLa) were studied using MTT assay. The MTT is a colorimetric method that measures the reduction of yellow 3-(4,5-dimethylthiazol-2-yl)-2,5-diphenyl tetrazolium bromide by mitochondrial succinate dehydrogenase to purple formazan. Because only living cells catalyze cellular reduction, it is possible to quantify the percentage of living cells in a solution. This assay can accurately quantify as few as 950 cells.

The presence of Fe nanowires had no significant effect on the cell proliferation and viability. Even when the cells were exposed to high Fe nanowires concentrations of 10000 per cell for 72 h they showed cell viability of about 80%. See, for example, Song. M.-M, et al., 2010. Biomaterials, 31(7), pp. 1509-17, which is incorporated by reference in its entirety. Finally, iron oxide nanowires were added to mouse fibroblasts. See, for example, Safi, M. et al., 2011. ACS Nano, 5(7), pp. 5354-5364, which is incorporated by reference in its entirety. Toxicity assays evaluating the mitochondrial activity, cell proliferation, and production of reactive oxygen species showed that the wires did not display acute short-term (<100 h) toxicity toward the cells.

TABLE 6
Cytotoxic effects of magnetic nanowires to cells in culture.

| | | | Nanowires to cells | |
|---|---|---|---|---|
| | | | concentration; cell | |
| | Nanowires | Nanowires | culture time with | |
| Reference | material | dimensions | nanowires | Cell line |
| Fung et al. | Nickel | D = 198-280 nm | Not mentioned | NIH-3T3 (mouse |
| 2008 | | L = 3-6 μm | | fibroblast) |
| Byrne et al. | | D = 200 nm | 110:1; 10 h | THP-1 (human |
| 2009 | | L = 20 μm | 500:1; 24 h | leukemia) |
| Song et al. | Iron | D = 50 nm | 10000:1; 72 h | HeLa (human |
| 2010 | | L = 1-10 μm | | cervical cancer) |
| Safi et al. | Iron oxide | D = 200 nm | 250:1; 24 h | NIH-3T3 (mouse |
| 2011 | | L = 1-40 μm | | fibroblast) |

D = diameter; L = length. [symbol]
/ [symbol] → cell viability over/below 80%

These studies are of remarkable importance because they establish working concentrations for nanowires of a particular material and size applied on certain human cell line with no cytotoxicity effects. Also, they gave potential suitability of both Ni and Fe nanowires for biological and, most importantly, clinical applications.

One way in which nanoparticles can target cancer cells is via the attachment of functional ligands that allow their interaction with biomolecules; this process is called biofunctionalization and makes nanostructures bioactive.

Nanomaterials have been designed with chemically modifiable surfaces to attach a variety of ligands that can turn these nanomaterials into bio-sensors, molecular-scale fluorescent tags, imaging agents, targeted molecular delivery vehicles, and other useful biological tools. See, for example, McNeil, S. E., 2005. Nanotechnology, 78 (September), pp. 585-594, which is incorporated by reference in its entirety. When targeting cancer cells, the approach differs significantly when working in vitro or in vivo mainly because of the completely different microenvironments and cellular organization levels in each case.

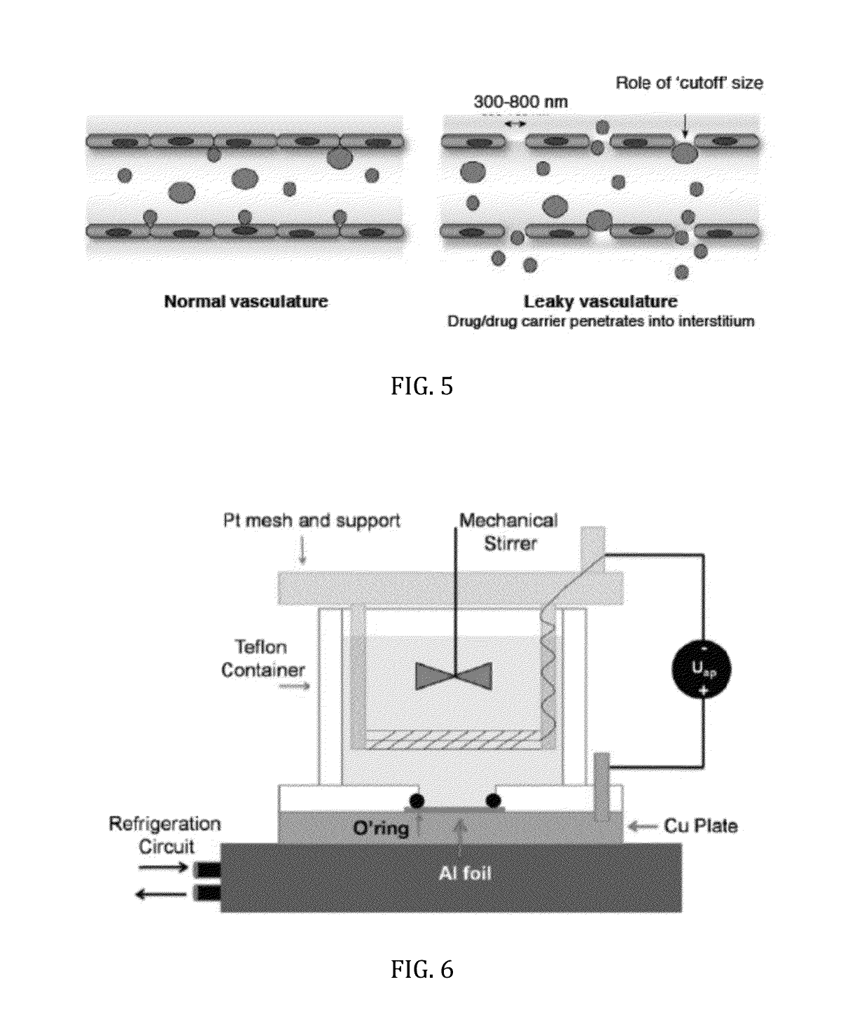

There are two ways of targeting cancer cells in vivo: passively and actively. Passive targeting takes advantage of the size of the nanostructures and the characteristics of the tumor vasculature: the enhanced permeability and retention (EPR) effect. Angiogenesis, the process of formation of new blood vessels, is a crucial feature in tumor development and metastasis. Tumor tissues, contrary to normal ones, can have gaps up to 800 nm between endothelial cells where the nanoparticles can extravasate (FIG. 5). See, for example, Song, M.-M. et al., 2010. Biomaterials, 31(7), pp. 1509-17, which is incorporated by reference in its entirety. However, with passive targeting there is not a homogeneous distribution of the nanoparticles in the tumor unless the circulation time of the nanoparticles is long enough to enhance their uptake into the tumor.

Active cell targeting, which works in vitro and in vivo, is known as antibody-based cancer therapy. See, for example, Glennie, M. J. & Johnson, P. W. M., 2000. Immunology today, 21(8), pp. 403-10; Carter, P., 2001. Nature Reviews cancer, 1 (November), pp. 118-29; Adams, G. P. & Weiner, L. M, 2005. Nature Biotechnology, 23(9), pp. 1147-57; and Oldham, R. K. & Dillman, R. O., 2008. Journal of Clinical Oncology, 26(11), pp. 1774-7, each of which is incorporated by reference in its entirety.

Since 2000 there have been clinical trials with monoclonal antibodies (mAb) approved by the US Food and Drug Administration (FDA). mAb binding to tumors has been shown to induce a number of signaling events that might play a role in controlling tumor growth. That is not surprising since the mAb could block important ligand-receptor interactions normally responsible for promoting tumor growth. See, for example, Glennie, M. J. & Johnson, P. W. M., 2000. Immunology today, 21(8), pp. 403-10; Carter, P., 2001. Nature Reviews Cancer, 1 (November), pp. 118-29, which is incorporated by reference in its entirety. Other works focus on ways of enhancing antibody-based therapy as it shows better results on non-solid (lymphoma and leukemia) over solid tumors, which suggested a better accessibility and targeting efficiency. Enhancement is suggested to be achieved via conjugation, vascularity targeting and mAb mixing. See, for example, Carter, P., 2001. Nature Reviews Cancer, 1 (November), pp. 118-29, which is incorporated by reference in its entirety.

All mentioned studies find antibody-based cancer therapy very promising because in the last 25 years new technologies have emerged to overcome the main limitations of mouse mAb immunogenicity of these foreign proteins in patients. These core technologies, in historical order of development, are chimerization and humanization of mouse antibodies (Carter 2001). There are several methods for antibody conjugation in biological applications. In an interesting cell separation study an antibody against mouse endothelial cells was conjugated to the Ni nanowire surface via self-assembled monolayers and chemical covalent reactions. See, for example, Gao, N., Wang, H. & Yang, E.-H., 2010. Nanotechnology, 21(10), pp. 1-8, which is incorporated by reference in its entirety. In other studies proteins were attached to nanowires made of or coated with gold by introducing a sulfhydryl group (—SH) to the protein that reacts with gold forming a S—Au bold, which is moderately strong. See, for example, Wildt, B., Mali, P. & Searson, P. C., 2006. Electrochemical template synthesis of multisegment nanowires: fabrication and protein functionalization. Langmuir the ACS journal of surfaces and colloids, 22(25), pp. 10528-34, Rozhkova, E. a. et al., 2009. Journal of Applied Physics, 105(7), pp. 105-7; Kim, Dong-Hyun et al., 2010. Nature Materials, 9 (November 2009), pp. 165-71; Frischmann, P., 2003. DTPA—Immobilize oligos to gold surfaces by multiple thiol anchorages, and Love, J. C. et al., 2005. Self-assembled monolayers of thiolates on metals as a form of nanotechnology, each of which is incorporated in its entirety. The activity of the modified antibody was preserved. See, for example, Kim, Dong-Hyun et al., 2010, Nature Materials, 9 (November 2009), pp. 165-71, which is incorporated in its entirety. This concept of antibody conjugation with magnetic microparticles has been proven recently to successfully target cancer-cells. In terms of the targeting moiety or antibody, its affinity for the surface of a predetermined cell type refers to the targeting moiety configured to have specific recognition and binding to the surface of the predetermined cell type, much like a mAB recognizes and binds to the target against which it was raised.

### Examples

### Synthesis

To ensure surface homogeneity the aluminum substrate was electropolished in 25% perchloric acid and 75% ethanol solution for 4 min at 25 V. For the first anodization, 0.3M oxalic acid was placed in the anodization cell. Oxalic acid temperature was kept at 4° C. and a constant voltage of 40V was applied for 24 hours (FIG. 6). The sample was then removed from the anodization cell. Alumina from the anodized sample was removed by immersing it in a chromium oxide and phosphoric acid aqueous solution at 30° C. for 12 hours with magnetic agitation. The same setup and parameters as for the first anodization were used for the second anodization except the duration of the second anodization was varied depending on the anticipated pore length (pore length growth rate: 2.5 μm/h). The sample was kept in the anodization cell for the alumina thinning process (creation of dendrites at the bottom of the pores). A step-like decreasing voltage starting at 40V and finishing at 5V was applied for an hour. The same solution and temperature values from anodization were used.

An electroplating solution (preparation recipe can be found in table 5) was added to the electroplating cell. The solution was kept at 40° C. during the electroplating process. Anodization and electroplating cells differ only on the size of the hole that faces the Al sample. Hole diameter of the electroplating cell was 7 mm.

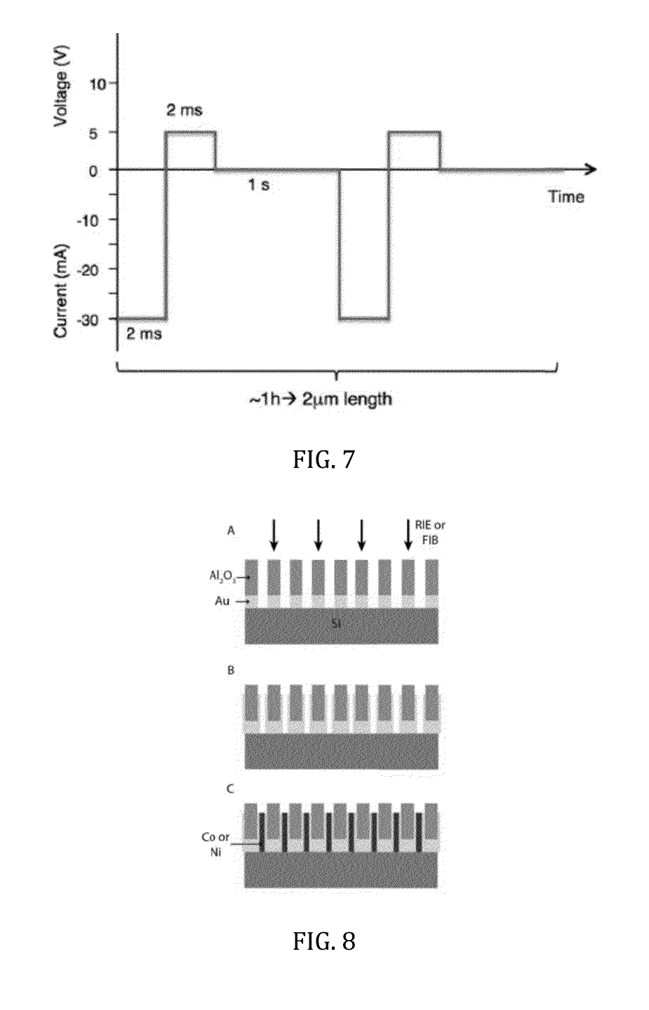

The applied voltage/current profile during electroplating is related to the deposition rate of the metal contained in the solution. The electroplating profile used is shown in FIG. 7.

During the first pulse, deposition occurred. A constant negative current pulse (Iₚᵤₗₛₑ) was applied to attract the positive metal ions. The current value depends both on the deposited material and the recipe of the solution. A current of −30 mA for 2 ms was applied. The second pulse discharged the alumina barrier-layer which acts as a capacitor. A pulse of voltage (Vₚᵤₗₛₑ=5V) was applied. By inverting the polarity, the pores were filled with fresh electroplating solution. This pulse may also help repairing small cracks that can appear during the first pulse. Next a recovery time occurred where neither Lₚᵤₗₛₑ nor Vₚᵤₗₛₑ was applied. This time allows the recovery of the ion concentration and pH at the bottom of the pores resulting in a more homogeneous deposition.

After electroplating, the wires were embedded in the alumina membrane. In order to release them by dissolving the membrane, the sample was immersed in 20 ml of 1M sodium hydroxide (NaOH) for 1 hour or until the black area of the electrodeposited region is seen fully detached. An alternative releasing process consists of immersing the sample overnight in chromsolution at 30° C. Gently tapping the construct causes a “cloud” of wires detaching from the substrate.

Finally the wires were cleaned prior adding them to the cells in culture. First, NaOH including the wires was transferred to a falcon tube. Using a moderately strong magnet the wires were concentrated at the bottom of the tube and everything but approximately 1 ml of the solution is removed. Then, the volume with wires was transferred to an eppendorf tube. Using DynaMag™ magnet (Invitrogen), the NaOH was replaced by MilliQ water 10 times mixing by pipetting in between the washing steps. Wires were kept in DI water for 24 h. Later, using DynaMag™ the wires were resuspended in 1 ml sterile PBS or cell culture medium as needed.

By determining the average hexagon length on the alumina membrane and knowing the total electroplated area, the total number of wires fabricated in one sample can be estimated. Assuming not many wires are lost in the previously mentioned cleaning process, this is the number that was resuspended in 1 ml of cell culture medium. By doing serial dilutions the desired number of wires was obtained. This desired number would exclusively depend on the cell line to which the wires will be added.

Coating Nanowires with Gold

Coating the nanowires with gold can make them biocompatible (Shukla 2007; Anon 2003). See, for example, Shukla, R., 2007. National Centre for Cell Science and Anon, 2003, Materials World Magazine, pp. 12-14, each of which is incorporated by reference in its entirety. Gold is an inert metal widely used in a broad repertoire of medical procedures such as reconstructive surgery of the middle ear, upper lid closure in facial nerve paresis-induced lagophthalmos, drug delivery microchips, antitumor treatment, treatment of rheumatoid arthritis, use on the surface of voice prostheses and endovascular stents. See, for example, Demann, Stein & Haubenreich, 2005. Gold as an implant in medicine and dentistry. Journal of Long-Term Effects of Medical Implants, 15(6), pp. 687-98, which is incorporated in its entirety. Additionally, in order to attach the antibodies to the nanowires the last ones need to be coated with gold. Coating with gold can be included in the fabrication process either before electroplating the magnetic metal or after dissolving the membrane.

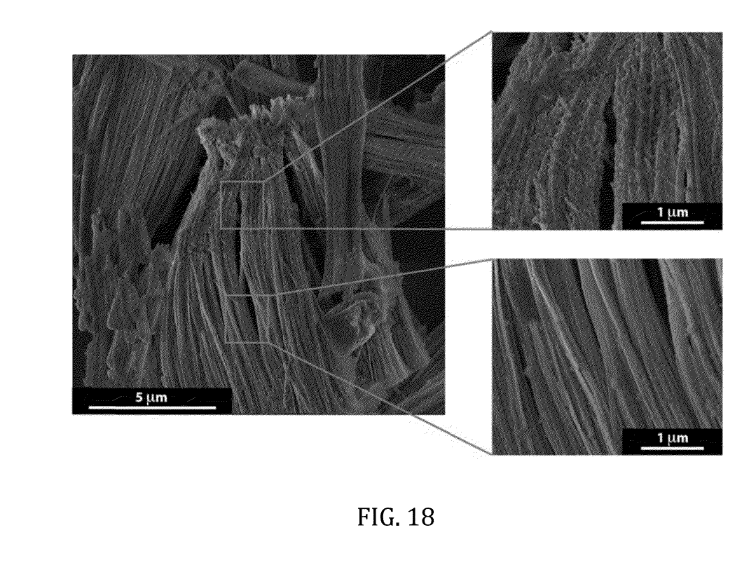

Sputtering gold on partially freestanding wires after dissolving the PAA showed a poor non-uniform gold coating (FIG. 15). However, when doing a pre-treatment on the nanowires's surface the gold coating was improved (FIG. 18). The pre-treatment consisted in using Ar/O₂ plasma to make the nanowires surface “hydrophilic-like” by introducing plenty of oxygen functional groups, like —OH, which will react with gold atoms in plasma condition and make the gold thin film more uniform on magnetic materials.

A specific way of electrochemical deposition of gold on PAA membranes leads to the formation of gold tubes inside the nanopores of PAA membranes. See, for example, Yang, G. et al., 2011. Clinica chimica acta; international journal of clinical chemistry, 412(17-18), pp. 1544-9, which is incorporated by reference in its entirety. For thick alumina membranes (e.g., >20 mm) the Al substrate is etched away and a thin 30 rim gold layer is sputtered in one of the sides of the PAA. After creating an electrical contact to the gold, in a solution containing 1% (w/w) HAuCl₄ and 0.1M perchloric acid, electrodeposition was performed by cyclic voltammetry at the scan rate of 100 mV/s and in the potential range between −300 and +500 mV. Gold nanotubes were synthesized with varied deposition times of 3, 5, 7, 8, 9, and 10 min, respectively. Yang et al used PAA of 200 nm diameter pore size and for a 3 min deposition time they reported an inner diameter of 180 nm, which means the thickness of the deposited gold is 20 nm. See, for example, Yang, G. et al., 2011. Clinica chimica acta; international journal of clinical chemistry, 412(17-18), pp. 1544-9, which is incorporated by reference in its entirety. After having the gold nanotubes, the magnetic material can be electrodeposited and then the membrane is dissolved having as a result the gold-coated wires in solution.

One drawback of this method is that very thick alumina membranes must be used, which implies long second anodization times (8 hours) and long NaOH submerging times to completely dissolve the PAA membrane. The method can be modified wherein the substrate was a silicon wafer with a thin film of Au on top of it and A1 sputtered on top of the Au (FIG. 8a). The entire Al was converted into PAA via anodization (FIG. 8b). After that, the Au without PAA on top was removed using reactive ion etching (RIE) or focused ion beam (FIB) (FIG. 8c). The top of the PAA was damaged during the etching process but the nanowire fabrication was not affected. After, the standard protocol for chemically electrodepositing Au into the nanopores to get nanotubes was applied. See, for example, Yang, G. et al., 2011. Clinica chimica acta; international journal of clinical chemistry, 412(17-18), pp. 1544-9, which is incorporated by reference in its entirety. Finally, the magnetic metal was electrodeposited resulting in Au coated magnetic nanowires.

Electroless gold deposition offers another possibility of coating the nanowires with gold. This type of deposition does not include electricity. It relies only on chemical reactions so the whole substrate surface is covered, which is advantageous when dealing with nanopores. Briefly, it consists of coating the substrate with a synthetic polymer, polyvinylpyrrolidone (PVP), submerge the substrate in, firstly, an SnCl₂ solution to have Sn²⁺ binding sites, secondly, in an AgNO₃ solution where the Ag ions will bind to the Sn²⁺, and, finally, in a Na₃Au(SO₃)₂ solution where by chemical principle, the more noble metal (Au in this case) will take the place of the less noble one (Ag). See, for example, Menon, V. P. & Martin, C. R., 1995. Fabrication and evaluation of nanoelectrode ensembles. Analytical Chemistry, 67(13), pp. 1920-1928.

Biofunctionalization with antibodies occurs through a chemical modification of the amino groups of the antibody, which has been successfully accomplished, —SH binds to the protein that reacts with gold forming a S—Au bond, which is moderately strong. See, for example, Wildt, B., Mali, P. & Searson, P. C., 2006. Electrochemical template synthesis of multisegment nanowires: fabrication and protein functionalization. Langmuir the ACS journal of surfaces and colloids, 22(25), pp. 10528-34, Rozhkova, E. a. et al., 2009. Journal of Applied Physics, 105(7), pp. 105-7; and Kim, Dong-Hyun et al., 2010. Nature Materials, 9 (November 2009), pp. 165-71, each of which is incorporated in its entirety.

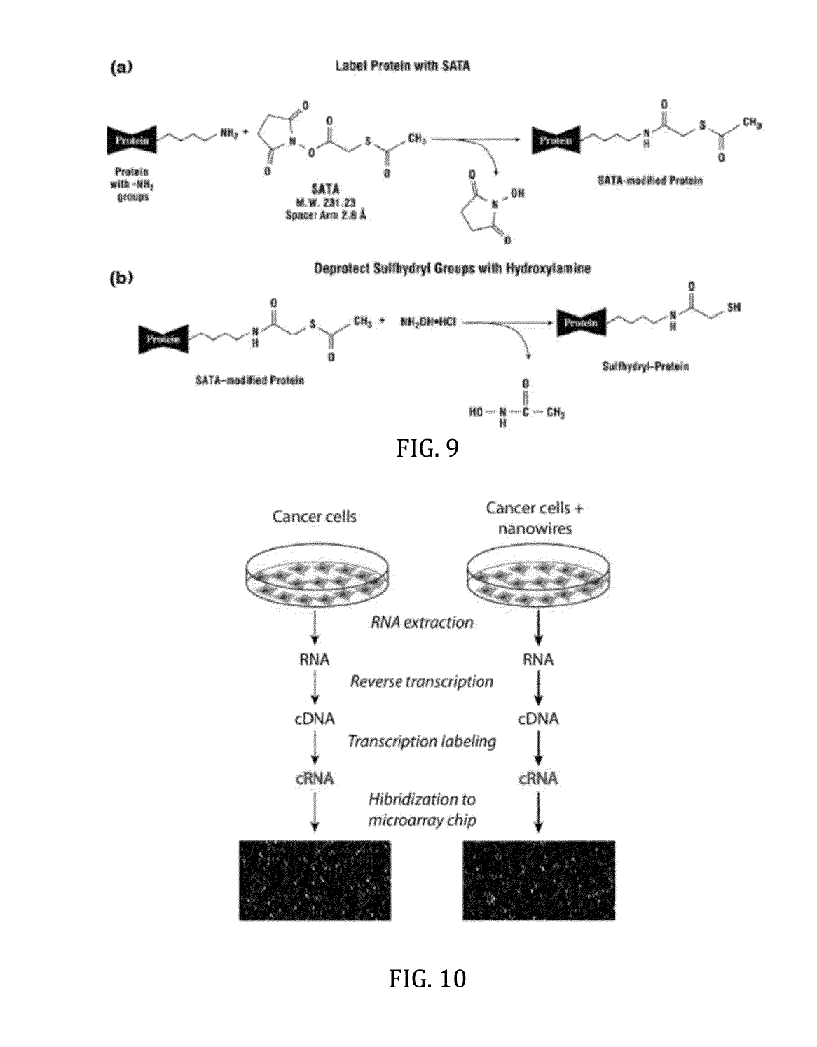

The antibody modification in detail is shown in FIG. 9. Firstly the antibody was incubated with N-succinimidyl S-acetylthioacetate (SATA; Thermo Scientific). SATA formed a covalent amide bond with primary amines of the antibody. The SATA-modified antibody contained a protected sulfhydryl group (FIG. 9a). After that, the modified antibody was incubated with hydroxylamine.HCl to deprotect the sulfur, resulting in a sulfhydryl group (—SH; FIG. 9b). This sulfhydryl formed a S—Au bond when incubating with Au-coated nanowires. In terms of the antibody or any targeting moiety having an affinity for a nanowire, the targeting moiety is configured to conjugate to the nanowire through the chemical groups, here, —SH groups of the antibody with the surface of the nanowire.

ELISA was used as a detection method for the functionalization reaction. The negative control consisted of nanowires with secondary antibody and the positive control just the antibody (without any modification) with the secondary antibody. To avoid non-specific binding of secondary antibodies to the nanowires, after incubation of the wires with the sulfhydryl-antibody the complex was incubated with bovine serum albumin (BSA).

Six human cancer cell lines were chosen for which there is evidence of overexpression of a membrane receptor (Table 7). These cell lines were tested in order to determine the following the harmful nanowire concentration for each cell line when a magnetic field is applied; variation between cell lines in that concentration; and whether that concentration differs when using Au-coated or non-Au coated nanowires.

TABLE 7
Cancer cell lines that will be tested with magnetic nanowires.

| Cancer | | | Raised | | |
|---|---|---|---|---|---|
| type | Cell line | Antibody | in | Isotype | Target |
| Ovarian | SKOV-3 | anti-ErbB2 | Mouse | IgG1 | ErbB2 |
| Breast | SKBR-3 | anti-ErbB2 | Mouse | IgG1 | ErbB2 |
| | BT-474 | | | | |
| Colon | HCT-116 | anti-EGFR | Rat | IgG2a | EGFR |
| Glioma | N10 | anti-IL13□2R | Rat | IgG2a | IL13□2R |
| Lymphoma | RL | anti-CD20 | Mouse | IgG2a | CD20 |

All antibodies are monoclonal (Abcam). ErbB2 (a.k.a. HER2/neu): Human Epidermal growth factor Receptor 2; EGFR: epithelial growth factor receptor: IL13G [symbol] 2R: interleukin 13 [symbol] 2 receptor; CD20: B-lymphocyte antigen CD20

Each one of the cell lines had been cultured as recommended by the vendor in sterile conditions. They were plated in 12-well plates with an initial suitable seeding density depending on each cell line. After 24 hours of culture different nanowire concentrations were added in such a way that the cell to wire ratio was on average 1:100, 1:500, or 1:1000. The cells were put back at the incubator and left there for different times: 6 h, 12 h, 24 h and 48 h. At each specific time point RNA was extracted from the cells using the RNeasy mini kit (Qiagen) with direct lysis. RNA was stored at −20° C. and handled to KAUST genomics core facility for microarray analysis. All six cancer cell lines were tested with both Au-coated and non-Au coated nanowires.

Determining the nanowire concentrations for which cancer cells in culture start to die without any specific targeting and the biological functions activated in the process, will help in designing therapies involving nanoparticles with fewer side effects.

### Genome-Wide Transcriptome Measurements

In order to assess the nanowires' effects on the cancer cells at the molecular level to understand if the presence of wires can activate a physiological defense response by the cells, sets of genes involved in a particular biological process or pathway that were differentially expressed when different nanowires' concentrations were added will be identified, establishing which genes were differentially expressed (up/down) at a given wire concentration. These measurements were done with both Au and non-Au coated nanowires to compare the changes of gene expression of both types of wires.

A one-color microarray-based gene expression protocol (Agilent Technologies 2009) was used on the extracted total RNA of the different cancer cell lines. The workflow for preparing the experiment is shown in FIG. 10. The microarray chip targets 27,958 human Entrez Gene RNAs; it has 9990 replicates of the biological probes; and its positive controls include 120 External RNA Control Consortium (ERCC) probes and 320 spike-in control probes. An RNA spike-in is an RNA transcript used to calibrate measurements in a DNA microarray experiment.

A single color array was used despite the fact of having two conditions mainly to avoid competition between the two florescent dyes cyanine 3 (Cy3) and cyanine 5 (Cy5). Cy3 was used because Cy5 is more prone to photobleaching.

FIG. 12 shows in the very first step the RNA extraction (RNeasy mini kit from Qiagen) on cancer cells under different conditions. RNA was extracted instead of DNA because it is more sensitive to changes in the environment and directly related to the expression or suppression of genes. The total RNA underwent a reverse transcription using a poly T primer to end up having cDNA. cDNA worked as a template to synthesize, label and amplify cRNA. From the total RNA only a part of it, the mRNA coded for proteins. This mRNA was characterized by having a poly adenine (A) tail on its 3′ side. The T7 RNA polymerase used for this transcription incorporates cytosine nucleotides already conjugated with Cy5. After that the cRNA was purified using a Qiagen column and finally was incubated with the microarray chip for 17 hours at 65° C.

The cRNA-DNA hybrid was much more stable than cDNA-DNA hybrid; hence, cRNA was used instead of cDNA as a target to hybridize the cDNA probes on the chip. Also, studies indicate that the labeling efficiency is much higher in cRNA compared to cDNA. See, for example, Bangarusamy, D. K., 2011. Genomics core facility. King Abdullah University of Science and Technology, which is incorporated in its entirety. T7 RNA polymerase, which was used to transcribe RNA from cDNA, was very specific to 17 promoter and had an extremely low error rate. The only disadvantage of this is that even the non-specific binding will be more stable. However, the highly specific probes that were in the chip arrays help prevent this. The probes were quite unique for a particular gene of a species. After culturing the cells with nanowires for the desire time (6 h, 12 h, 24 h and 48 h) the total RNA was collected. Also, for each time point, RNA from control cells (no wires added) was extracted. Both RNA extractions were done by replicate for each time point.

Thereafter, each probe in the chip array has assigned an intensity value. Some steps are needed before treating the intensity data as expression values and do the correspondent analysis. First, the Agilent Feature Extraction software automatically performs background subtraction & LOWESS (locally weighted scatterplot smoothing) smoothing. Then, all intensity values need to be normalized. Results from different chips (including the replicates) are going to be compared for further analysis. So, normalization is done in order not to introduce bias in the comparisons. Next, a statistical test is done to determine how different the intensity is for the same probe in the replicate slide. A p-value is assigned to each probe wherein the lower the p-value, the more reliable the data. Afterwards a filtration of the gene list is done. Only genes with a p-vale<0.05 will be taking into account for the analysis. Next, a determination of fold changes is made. Fold (N) is a ratio. In this case is the ratio between the intensity value of a probe of the cells with nanowires and the control cells (only cell culture media added).

$$Fold change of cells + nanowires vs . control cells = \frac{{I}_{Cells + nanowires}}{{I}_{Control cells}}$$

A gene is going to be considered up-regulated every time N>2. Following the same logic a gene will be considered down-regulated whenever N<2.

Fold comparisons for up-regulated genes that will be included in the analysis are:
6 h cells+nanowires vs. 6 h control cells
12 h cells+nanowires vs. 12 h control cells
24 h cells+nanowires vs. 24 h control cells
48 h cells+nanowires vs. 48 h control cells

Next the data undergo gene ontology (GO) and pathway analysis. On the sub-set of genes that were either up or down-regulated GO and pathway enrichment analysis will be performed using the gene functional annotation online tool DAVID. All GO biological process terms and pathways with a p-value <10⁻³ were taken into account in the analysis. Finally, Gene clustering analysis done with DAVID compare genes expressed at the same time point. However, it would be useful to do a gene clustering for all four time points to determine if there are functional groups of genes highly expressed at early or late stages of adding the wires or there aren't at all. In order to do it a list with gene IDs and expression values for each time point will be uploaded to the free software MeV (Multi Experiment Viewer) and a gene clustering analysis including GO term will be performed.

### Biocompatibility Studies

Since the usage of nanostructures, and particularly magnetic nanostructures, is becoming more popular in the biomedical field, drug delivery, imaging, disease detection, etc., it is crucial to determine what are the biological effects, besides the intended function of a nanostructure, in vivo. Starting from a simplified approach, a panel of six cell lines was tested against a panel of magnetic micro and nanostructures (wires and disks) both coated and non-coated with gold (Table 8). Again, different concentrations of nanostructures were added to the different cell lines.

TABLE 8
List of magnetic micro and nano structure that will be tested
for biocompatibility on a panel of human cell lines.

| Magnetic | |
|---|---|
| nanostructures | |
| (both Au and | |
| non-Au coated) | Human cell type (cell line, vendor) |
| Co nanowires | Marrow stromal cells (MSC001F, StemCell tech.) |
| Ni nanowires | Fibroblasts (Wi-38, ATCC) |
| Permalloy microdiscs | Embryonic kidney cells (293FT, Invitrogen) |
| Permalloy nanodiscs | Ovarian cancer (SKOV3, ATCC) |
| | Breast cancer (BKBR3, ATCC) |
| | Lymphoma (RL, ATCC) |
| | Leukemia (THP-1, ATCC) |

A Vybrant® MTT Cell Proliferation Assay Kit (Invitrogen) was done according to the vendor's recommendations. After 24 h of seeding the cells the one of the magnetic nanostructures were added. Cells were incubated with the nanostructures for 24-48 h and then they were labeled and after adding SDS.HCl solution to make formazan soluble, the plate was read at 560 nm. Cell culture media was the blank, the positive control was cells without addition of nanostructures and the negative control was cells to which apoptosis was induced (30 min UV light).

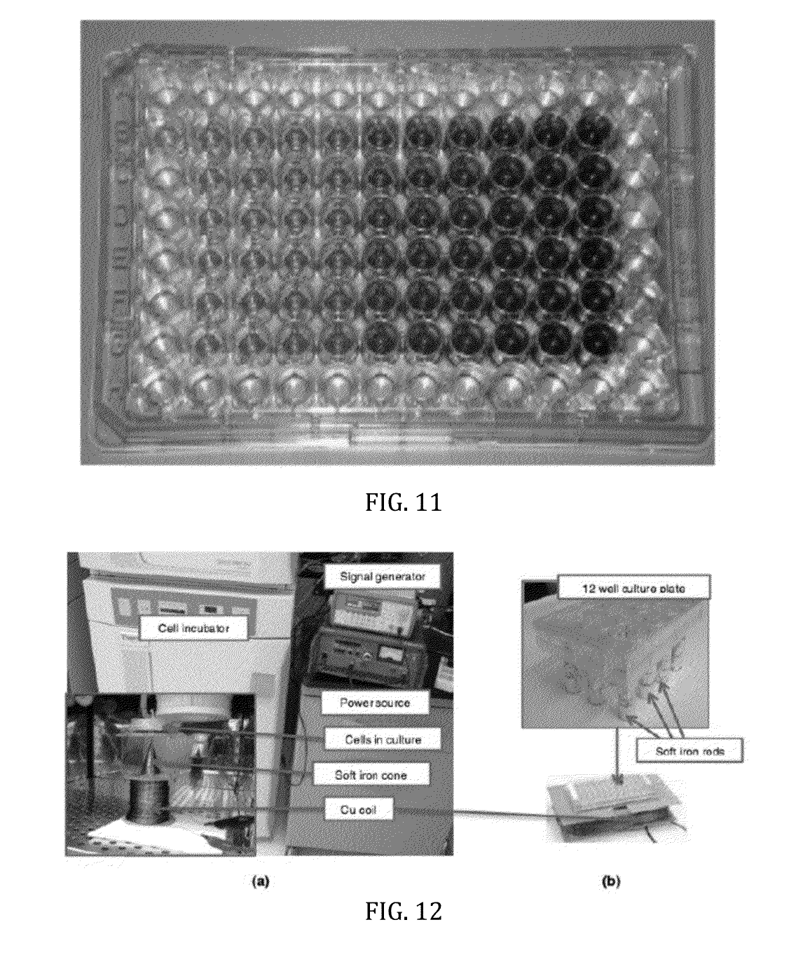

FIG. 11 shows how an MTT assay. On the right, all dark wells indicate the highest cell viability number while on the left, light coloration shows the opposite.

The targeting efficiency of the antibodies was corroborated using western blot on whole cell lysates. Also, immunohistochemistry showed the distribution of the antibodies on the different cell lines. After biofunctionalizing the nanowires with the antibodies, fluorescence secondary antibodies were used to corroborate that the antibody was coating on the wires.

TEM images that show the exact position of the nanowire on the cell membrane were taken before applying an external magnetic field. Also TEM was done on non-Au coated wires added to cells to check if the cells internalize the wires.

### Effects of Magnetic Field in Nanowires

Magnetic nanowires can be manipulated in various ways using magnetic fields. An inhomogeneous magnetic field exerts a translational force to Magnetic nanowires that can be used to move or confine them. Assuming a MNW with a magnetic moment in, the force F applied by a magnetic field H is

F=μ₀(m·∇)H, (4)

where μ₀ is the permeability of free space. If the magnetization of the Magnetic nanowires is not aligned with the direction of the applied field, a torque will be exerted on the wires causing them to align in a DC field or oscillate in a low-frequency AC field. The torque is given by

$$τ = {μ}_{0} m \times H = {μ}_{0} m H \cdot sin θ , \qquad ( 5 )$$

where θ the angle between in and H. In the case of full alignment, where H and m are parallel (θ=0), there is no torque, and in the case where H and m are perpendicular, (θ=90) the torque reaches a maximum value.

A property of magnetic nanowires that has been exploited in biomedical applications is the thermal losses produced during a magnetization cycle (equivalent to the area of the magnetization curve). The work done by the external field during the alignment of the magnetic moments produces heat, which is dissipated to the surrounding media thereby elevating its temperature. For ferromagnetic particles, the amount of heat generated (P) per unit volume is given by the frequency (f) multiplied by the area of the hysteresis loop Pankhurst, Q. a et al., 2003. Applications of magnetic nanoparticles in biomedicine. Journal of Physics D: Applied Physics, 36(13), p. R167-R181.

P=μ₀f [symbol] HdM, (6)

where H is the strength of the applied AC magnetic field and M the magnetization. As indicated by equation 3, the heat produced increases with the strength of the magnetic field applied as well as with the field's frequency. One final property of magnetic nanowires is magnetostriction, which was mentioned in this document previously.

To measure the value of in a vibrating sample magnetometer (VSM) measurement was done on a small area (˜mm²) of an array of wires. The value given by the VSM included the magnetic moment and magnetostatic interactions among the wires. However, an approximation was done and mₛ was divided by the total number of wires to give the maximum magnetic moment of a single wire and therefore determine the maximum torque exerted on a single wire within a given field. To estimate the magnitude of the force a cell feels when a wire is attached to it, the torque must be divided by the lever (half of the wire length to have a maximum value, assuming the wire is attached the cell by its mid-point).

TABLE 9
Estimation of torque exerted on a single wire and the magnitude
of the force the wire transmit to the cell once attached to it.

| | Pore | Nanowire | | Torque | |
|---|---|---|---|---|---|
| | diameter | length | mₛ | (×10⁻¹⁷ | Force |
| Material | (nm) | (μm) | (μemu) | N · m) | (pN) |
| CoNi | 35 | 1 | 961.6 | 1.6 | 33 |
| CoNi | 35 | 2 | 544.7 | 1.5 | 16 |
| CoNi | 55 | 2 | 624.6 | 1.1 | 12 |

Table 9 shows a summary of both the torque exerted on the wire by a magnetic field and the force exerted to the cell.

Fung et al 2008 reported a torque of 1.3×10⁻¹⁴N·m for a Ni nanowire of 198-280 nm diameter and 3-6 μm length under a field 250 mT (our calculations were made with a field of 100 mT). See, Fung, A. O. et al., 2008. Induction of Cell Death by Magnetic Actuation of Nickel Nanowires Internalized by Fibroblasts, The Journal of Physical Chemistry C Letters, 112, pp. 15085-15088, which is incorporated in its entirety. Kim et al 2010 reported maximum forces of 53 pN of magnetic microdisks of 1 μm diameter made of permalloy that effectively destroy cancer cells. Kim, Dong-Hyun et al., 2010. Nature Materials, 9 (November 2009), pp. 165-71, which is incorporated by reference in its entirety.

The first one was made to determine the needed force to disrupt cell membrane using chemically modified AFM probes. They reported a value of 450±22 pN which is one order of magnitude above the forces exerted by nanowires. In the second study the authors used an optical trap to measure the magnitude of the force generated by actin filament polymerization which was around ˜2 pN. Previous studies give an idea of how strongly the nanowires forces will be compared to forces within cells. They will not be able to disrupt the plasma membrane but if internalized they might affect the cells. See, for example, Afrin, R., Yamada, T. & Ikai, A., 2004. Analysis of force curves obtained on the live cell membrane using chemically modified AFM probes. Ultramicroscopy, 100(3-4), pp. 187-95, and Footer, M. J. et al., 2007. Direct measurement of force generation by actin filament polymerization using an optical trap. Proceedings of the National Academy of Sciences of the United States of America, 104(7), pp. 2181-6, each of which is incorporated in its entirety.

### Experimental Setup

The cells were cultured as normally in 12, 24 or 96 wells plates. After 24 h the nanowire-antibody complex was added. The effects on the cells without external magnetic field was assessed with the MTT assay. Using the experimental setup shown in FIG. 12, an alternating magnetic field was applied on the cells-nanowire-antibody complex. The exposure time, field intensity and frequency were tuned accordingly but starting values will be 10, 20, 30 mins; 30, 60, 90 Oersted; 20, 40, 60 Hetrz respectively. See, for example, Kim, Dong-Hyun et al., 2010. Nature Materials, 9 (November 2009), pp. 165-71, which is incorporated by reference in its entirety.

In order to apply an alternating magnetic field a power source and a signal generator were used. Current was passed through several turns of copper coil. A soft iron cone (FIG. 12a) or rods (FIG. 12b) was used to concentrate the field underneath the cell culture plates. The inset in FIG. 12a shows what is inside the cell incubator (note that the cables that are attached to the Cu coil are connected to the power source). The very same experimental setup from FIG. 12a can be used when only one condition is considered. However, when dealing with different cell lines and different nanowires concentration, materials and dimensions, 12, 24 or even 96 well plates (FIG. 12b) will be needed.

After field exposure a membrane integrity assay and TUNEL (terminal deoxynucleotidyl transferase dUTP nick end labeling) apoptosis assay were applied on the cells to determine if the cell membrane and DNA were disrupted. Microarray experiments were also done to corroborate if the set of up regulated genes in the process correspond to genes involve in apoptosis or other stress related cellular pathways.

### Results

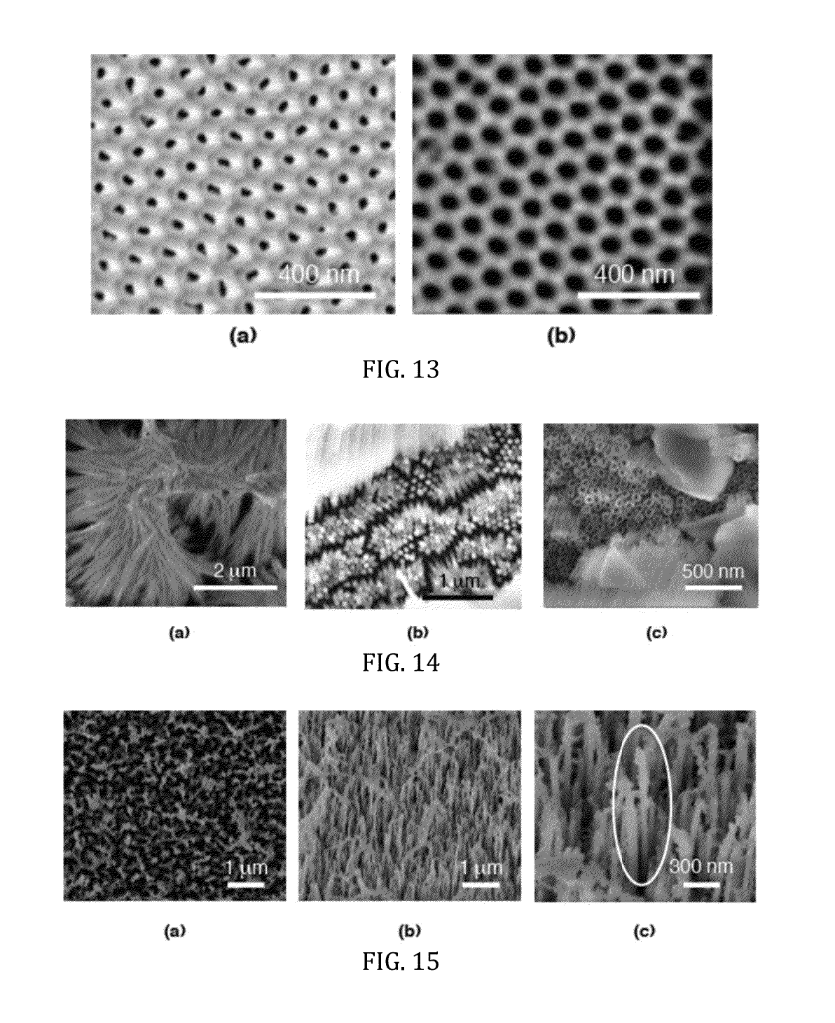

FIG. 13 shows SEM pictures of two alumina nanomembranes with different pore diameter (PD) and interpore distance (ID) values. For both cases the same electrolytic bath was used (0.3M oxalic acid) under the same voltage, time and temperature conditions. For the alumina membrane shown on (b) the pores were widened via isotropic chemical etching (0.3M oxalic acid at 30° C. with agitation for 2 h). It can be observed from both pictures an overall nice pore size homogeneity.

When submerging the wires in 1M NaOH the alumina membrane dissolution was not isotropic. Separate patches of wires were detached at different moments; perhaps because the membrane itself did not have a smooth surface.

SEM pictures were taken after submerging the sample on NaOH for 1 h and a mesh of wires was seen (FIG. 14a). Because of the surface tension of the liquid NaOH, the wires did not stand but bended while the NaOH dried out. To avoid this, the sample was dryed in a controlled manner using critical point drying (CPD). It was submerged for only 2 minutes in NaOH and dried out using CPD (FIG. 14b). Then submerged again for 2 minutes with another round of CPD (FIG. 14c).

As seen in FIG. 14 when CDP was applied, the nanowires remained standing. However, in FIGS. 14b and 14c, besides the standing wires alumina membrane regions are present. This is a clear indication that the dissolution of the alumina membrane with NaOH is highly anisotropic. In picture 15c instead of wires tubes were observed. It was not possible to conclude if what is shown were the wires themselves or still the alumina membrane. In trying to get a more uniform dissolution of the alumina membrane a sample of Ni nanowires was submerged in NaOH for 4 minutes with constant manual shaking. It was dried out using CPD and gold was sputtered to try to obtained a thin film of 5 nm thickness.

FIG. 15 shows the SEM pictures of a Ni sample. No alumina membrane regions were found in this sample which clearly indicated that this time the membrane was dissolved uniformly as seen in FIGS. 15a and 15b. Regarding the gold coating FIG. 15c shows a close up with standing wires. The wire standing in the center of FIG. 15c (circled in white) is partially covered with gold granules instead of a smooth gold layer. This happened first of all because of the complex geometry of the substrate and secondly because gold is a very inert element which apparently reacts easily with gold rather than with nickel. The gold coating process of the nanowires has to be optimized.

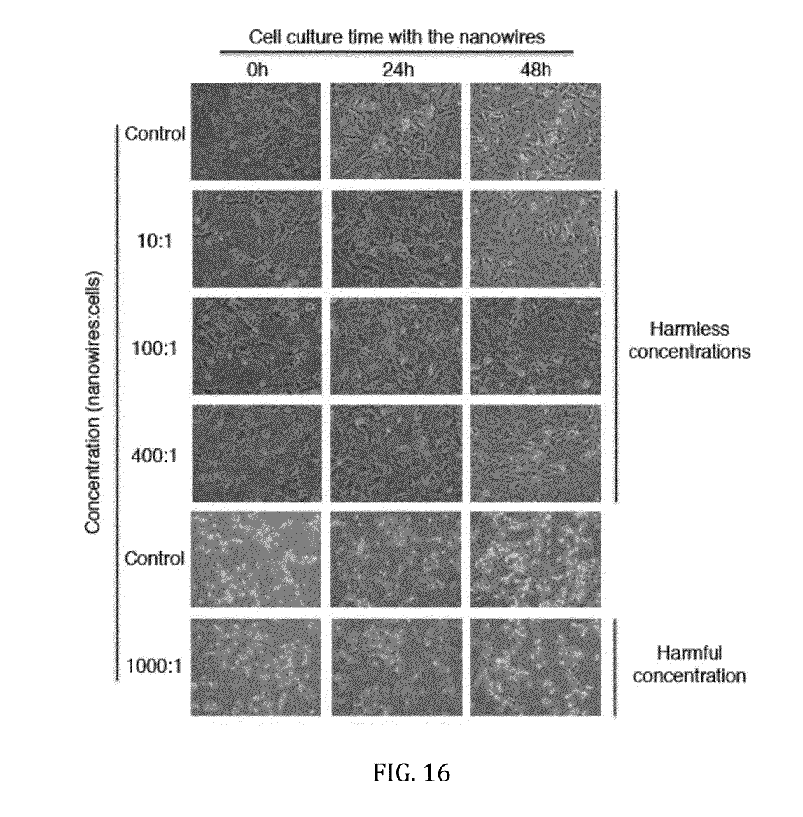

After growing ovarian cancer cells (SKOV3) for 24 h, NiCo nanowires were added. In this experiment, the nanowires were not coated with gold and no magnetic field was applied. RNA extraction was done on the cells with concentrations of 1000 wires per cell (1000:1) and their negative controls (addition of fresh culture medium) for further genome-wide transcriptome measurements. It can be observed from FIG. 16 that cell grew normally except when trying a concentration of 1000 wires per cell and the culture times of 48 h. A low cell number (bottom right corner image) is seen if compared to the controls cells picture right above it.

Concerning nanowire concentrations that did not cause cell death (10, 100 and 400 wires per cells), even though no difference in the optical images was observed, transcriptomic measurements will be performed to establish how significant gene expression was altered.

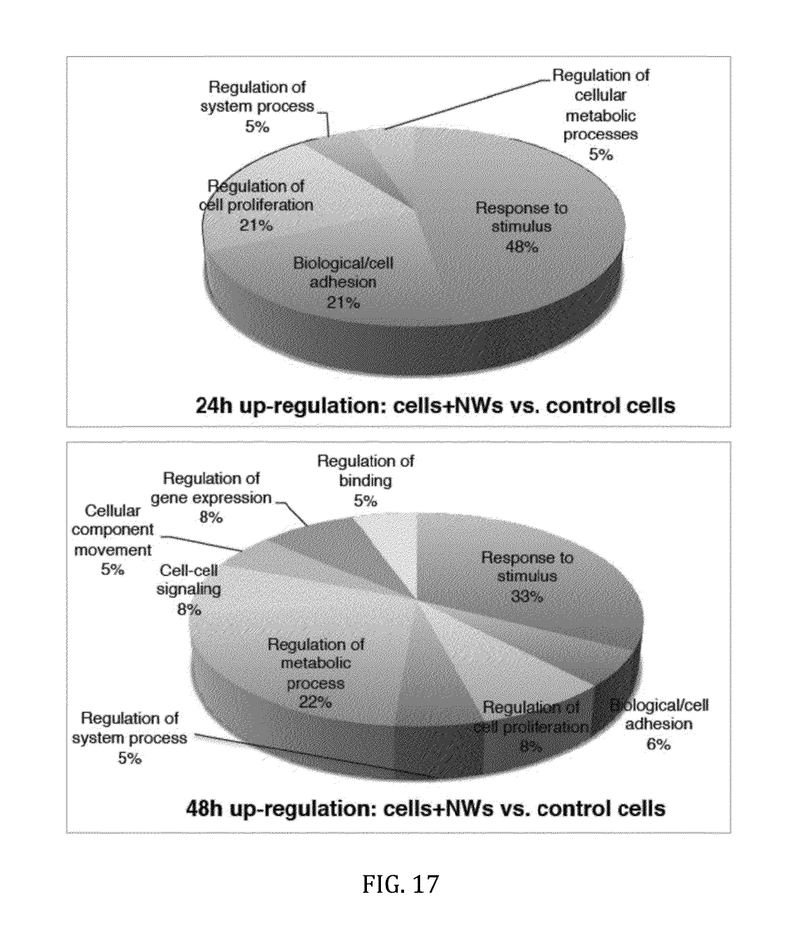

FIG. 17 shows the Gene Ontology (GO) enrichment analyses of differentially expressed genes performed using the DAVID gene functional annotation database. At the earliest time point (6 h) there was no gene expression change when comparing control cells with cells incubated with a concentration of 1000 nanowires per cell. At 12 h only 3 GO terms were upregulated all falling in the category of response to stimulus as expected. At 24 h and 48 h upregulation got more diverse as more terms were upregulated. All different categories are illustrated in two pie charts in FIG. 17. At 24 h besides the response to stimulus process other got enriched like biological adhesion, cell proliferation and metabolic processes. At 48 h the biological processes enrichment got more diverse reducing the portion of genes responsible for responding to stimulus and term like regulation of gene expression and an increase in metabolic processes regulation.

Other embodiments are within the scope of the following claims.

What is claimed is:

1. A composition comprising:
a plurality of nanowires, at least a portion of the plurality of nanowires being responsive to a magnetic field; and
a plurality of targeting moieties, each of the moieties having a first affinity for a surface of a predetermined cell type and a second affinity for a nanowire in the plurality of nanowires, wherein at least one moiety in the plurality of targeting moieties is in contact with a nanowire of the plurality of nanowires, wherein the plurality of nanowires responds to a magnetic field.

2. The composition of claim 1, wherein the first affinity includes the targeting moiety configured to have specific recognition and binding to the surface of the predetermined cell type.

3. The composition of claim 1, wherein the second affinity includes the targeting moiety is configured to conjugate to the nanowire through the chemical groups of the moiety with the surface of the nanowire.

4. The composition of claim 1, wherein each of the plurality of nanowires includes nickel, cobalt, iron, or alloys or combinations thereof.

5. The composition of claim 1, wherein each nanowire in the plurality of nanowires includes a gold coating.

6. The composition of claim 1, wherein the targeting moiety is an antibody.

7. The composition of claim 6, wherein the antibody has a first affinity to a membrane receptor of a cancer cell.

8. The composition of claim 1, wherein the moieties are randomly distributed on a nanowire in the plurality of nanowires.

9. The composition of claim 1, wherein the moieties have a patterned distribution on a nanowire in the plurality of nanowires.

10. The composition of claim 6, wherein the antibody has a second affinity for a nanowire in the plurality of nanowires wherein the antibody is a chemically modified antibody.

11. The composition of claim 10, wherein the chemically modified antibody includes sulfhydryl groups.

12. The composition of claim 1, wherein at least a portion of the plurality of nanowires are magnetic.

13. The composition of claim 1, wherein at least a portion of the plurality of nanowires is magnetostrictive.

14. A method of making a composition comprising:
forming a plurality of nanowires wherein the diameter and length of each nanowire are controllable;
configuring the plurality of nanowires to be biocompatible;
contacting a plurality of targeting moieties with a plurality of nanowires, wherein the moieties have a first affinity for a surface of a predetermined cell type and a second affinity for a nanowire in the plurality of nanowires, wherein the plurality of nanowires responds to a magnetic field whereby a force is exerted on the predetermined cell type causing cell modification.

15. The method of claim 14, further comprising forming a plurality of nanowires wherein the diameter and length of each nanowire are predetermined before contacting a plurality of targeting moieties with a plurality of nanowires, wherein the moieties have a first affinity for a surface of a predetermined cell type and a second affinity for a nanowire in the plurality of nanowires, wherein the plurality of nanowires responds to a magnetic field whereby a force is exerted on the predetermined cell type causing cell modification.

16. The method of claim 15, further comprising configuring the plurality of nanowires to be biocompatible before contacting a plurality of targeting moieties with a plurality of nanowires, wherein the moieties have a first affinity for a surface of a predetermined cell type and a second affinity for a nanowire in the plurality of nanowires, wherein the plurality of nanowires responds to a magnetic field whereby a force is exerted on the predetermined cell type causing cell modification.

17. The method of claim 15, wherein forming the plurality of nanowires includes electrodeposition into nanopores of a membrane.

18. The method of claim 17, wherein the length of each nanowire in the plurality of nanowires is dependent on electrodeposition time.

19. The method of claim 17, wherein the diameter of each nanowire in the plurality of nanowires is dependent on the nanopore size of the membrane.

20. The method of claim 17, wherein the membrane is an alumina membrane.

21. The method of claim 20, wherein forming the alumina membrane includes an anodization process including a highly pure aluminum substrate.

22. The method of claim 20, wherein the alumina membrane has a honeycomb structure.

23. The method of claim 16, wherein configuring the plurality of nanowires to be biocompatible includes coating the plurality of nanowires with gold.

24. The method of claim 23, wherein coating the plurality of nanowires with gold includes electroless deposition of gold into nanopores of a membrane.

25. The method of claim 24, wherein the membrane is an alumina membrane.

26. The method of claim 14, wherein the targeting moiety is an antibody.

27. A method of cell modification comprising:
administering a composition including a plurality of nanowires, at east a portion of the plurality of nanowires being responsive to a magnetic field;
applying the magnetic field to the composition, and
modifying cell of the predetermined cell type, wherein a plurality of targeting moieties, each of the moieties having a first affinity for a surface of a predetermined cell type and a second affinity for a nanowire in the plurality of nanowires, wherein the at least one moiety in the plurality of moieties is in contact with a nanowire of the plurality of nanowires, wherein the plurality of nanowires responds to a magnetic field whereby a force is exerted on the predetermined cell type causing cell modification.

28. The method of claim 27, wherein applying the magnetic field includes subjecting the plurality of nanowires to a magnetic field strength effective to cause the plurality of nanowires to vibrate.

29. The method of claim 27, further comprising introducing the composition to the patient.

30. The method of claim 27, wherein applying the magnetic field includes generating the magnetic field inside the patient.

31. The method of claim 27, wherein applying the magnetic field includes generating the magnetic field external to the patient.
